SEQUENCE LISTING (70 sequences)

SEQ ID NO: 1 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 2 - protein, 281 aa, Thinopyrum elongatum
MATSTSTSTPIIFYDIAQRPPVAETCCAVNPWKSRLALNFKAVPYTTTWVKMPDISSVRASLNVPACRKFADGSDFNTLPIIHDPATDSLIGDSLDIAAYLQRTYPASGAGDLFPPQKLDYAVGRDMQQLLFPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 3 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAVYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 4 - protein, 281 aa, Thinopyrum ponticum
MATSASTSTPIIPTAIAGAPPVAGTCCAVAPTLSALALAPLAVPTTTTTVLMPAISSVAASLAVPACALPAAGSAPATLPIIHAPATASLVGASPAIAATLGATTLASGAGALPPPGLLATAVGAAMPGLLIPLSGIAASPGLAATAAPASAVAAAPTAHVGLMVHGLPLAPATAAVTLAGPVAAAGLSSTAALGMVGGAAALMMGSLAAMLGALAALPALAASGPPLLGGAATTAAMIVGGTLAMMAATLPVSGTGGAAACHGAIPGGLHAALALTAGVL

SEQ ID NO: 5 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIVAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 6 - protein, 281 aa, Thinopyrum ponticum
MATSASTSTPIIFYDIAQRPPVAETCCAVNPWKSRLALNFKAVPYTTIWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 7 - protein, 281 aa, Thinopyrum ponticum
MATSASTSTPIIFYDIAQRPPVAETCCAVNPWKFRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 8 - protein, 208 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSL

SEQ ID NO: 9 - protein, 242 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGG

SEQ ID NO: 10 - protein, 281 aa, Thinopyrum ponticum
MATSASTSTPIIFYDIAQRPPVAKTCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 11 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLISLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 12 - protein, 281 aa, Thinopyrum ponticum
MATSASTSTPIIFYDIAQRPPVAETCCAVNPWKSKLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 13 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSFVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 14 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASLELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 15 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIHASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 16 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYTVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 17 - protein, 281 aa, Thinopyrum ponticum
MATSASTSTPIIFYDIAQRPPVAETCCAVNPWKSRLALNFKAVPYTTTWVKMPDISSVRASFNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 18 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVIKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 19 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATVDVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 20 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPVSGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 21 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDVAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 22 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGVGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 23 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRRAGLSSWDDLEMVGEARDKMMQSLRNMLGDLAALFRKDASGPFLLGQRATYADMIVGGWLRMMRAMLPVSEWQEARACHGAIFGQLHDALDKYAEVK

SEQ ID NO: 24 - protein, 281 aa, Thinopyrum ponticum
MATSASTSTPIIFYDIAQRPPVAETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMPQLLIPLSEIRASPELADYARFNSNVDAAFTAHVGLMVHGLPLDPATADVTKAEFVRHAGLSSWDDLEMVGEARDKMMQSLRNMLGDLAALFRKDASGPFLLGQRATYADMIVGGWLRMMRATLPVSEWQEARACHGAIFGQLHDALDKYAEVK

SEQ ID NO: 25 - protein, 280 aa, Epichloe bromicola
MATSTSISTPIIFYDIAQRPPVTETCCAVNPWKSRLALNFKAVPYTTTWVKMPDISSVRASLNVPACRKFADGSDFNTLPIIHDPATDSLVGDSFDIAAYLQRTYPASGAGDLFPPQKLDYAVGRDMQLLIPLSETRASPELADYARFNSNVDAAFTAHVGLMVHGLPLDPATADVTKAEFVRRAGVSSWEDFEMVGEAREKMMQSLRNMLGDLAALFRRDASGPFLLGQKATYADLIVGGWLRMMRATLPASEWQEVRACHGAVFGQLHDALDKYAEVK

SEQ ID NO: 26 - protein, 279 aa, Epichloe amarillans
MATSTSTPIIFYDIAQRPPVTETCCAVNPWKSRLALNFKAVPYTTTWVKMPDISSVRASLNLPACRKFADGTDFDTLPIIHDPATGSLIGDSFDIAAYLQRTYPASGAGDLFPPQKLDYAAGRDTQLLIPLSEVRAASPELADYARFNSNVDAAFTAHVGLMVHGLPLDPATADVTKAEFVRRAAVSSWDDLDMVGDARDKMMQSLRNTLGDLAALFRRDASGPFLLGPKATYADLIVGGWLRMMRATLPPSEWQAARAWHGAVFGQLHDALDKYAEVK

SEQ ID NO: 27 - protein, 264 aa, Epichloe baconii
MATSTSTSTSTSTPIIFYDIAQRPPVTETCCAVNPWKSRLALNFKAVPYTTTWVKMPDISSVRASLNLPACRKFADGTDFNTLPIIHDPATGSLVGDSFDIAAYLQRTDTQLLIPLSEVRAASSDLADYARFNSNVDAAFTAHVGLMVHGLPLDPATADVTKAEFVRRAGVSSWDDFEMAGEAREKMMQSLRNTLGDLAALFRRDASGPFLLGRKATYADLIVGGWLRMMRATLPASEWQAARAWHGAVFGQLHDALDKYAEVK

SEQ ID NO: 28 - protein, 286 aa, Epichloe festucae
MATSTSTSTSTPIIFYDIAQRPPVTETCCAVNPWKTRLALNFKAVTYTTTWVKMPDISGVRASLNVPACRKFADGTDFNTLPIIHDPATGSLIGDSFDIAAYLQRTYPASGAGHLFPPLPPPQKLDYAVGRDMQLLIPLSEVRASSELADYARFNSNVDAAFTAHVGVMVHGLPLDPATADVTKAEFVRRAGVSSWEDFEMVGEAREKMMQSLRNMLGDLAALFRRDASGPFLLGQQATYADLIVGGWLRMMRATLPASEWQEVRAWHGAVFGRLHDALDKYAEVK

SEQ ID NO: 29 - protein, 281 aa, Epichloe gansuensis
MATSTSTSASTPIIFYDIAQRPPVTETCCAVNPWKSRLALNFKAVPYTTTWVEMPDISSVRASLNLPACRKFADGSDFNTLPIIHDPATGSLIGDSFDIAAYLQRTYPASGADDLFPPQKLDYVVGSHVQPFIPLSDIRASEFADYARFNSNVDAAFTAHVGLMLHGLPLDPATADVTKAEFVRRAGVSSWEDFEMVGEAREKMMQSFRTMLEDLAALFRRDATGPFLLGQKATYADLIVGGWLRMMRATLPASEWQEARAWHGAVFGQLHDALDKYAEVK

SEQ ID NO: 30 - protein, 281 aa, Epichloe typhina
MATSSTSTSTPIIFYDIAQRPPVAETCCAVNPWKSRLALNFKAVPYTTTWVKMPDISSVRASLNVPACRKFADGSDFNTLPIMHDPATDSLIGDSFDIAAYLQRTYPASGAGDLFPPQKLDYAVGRDMQLLIPLSEVRASPELADYARFNSNVDAAFTAHVGLMVHGLPLDPATADVTKAEFVRRAGVSSWEDFEMVGEVREKMMQSLRNMLGDLAALFRRDASGPFLLGQRATYADLIVGGWLRMMRATLPASEWQEARACHGAIFGQLHDALDKYAEVK

SEQ ID NO: 31 - protein, 281 aa, Epichloe uncinata; identical to SEQ ID NO: 30

SEQ ID NO: 32 - protein, 280 aa, Epichloe sylvatica
MTTSTSTSTPIIFYDIAQRPPVAETCCAVNPWKSRLALNFKAVPYKTTWVKMPDISSVRASLKVPACRKFADGSDFNTLPIMHDPATDSLLGDSFDIAAYLQRTYPASGAGDLFPPQKLDYAVGRDMQLLIPLSEVRASPELADYARFNSNVDAAFTAHVGLMVHGLPLDPATADVTKAEFVRRAGVSSWEDLEMVGEAREKMMQSLRNMLGDLAALFRRDASGPFLLGQRATYADLIVGGWLRMMRATLPASEWQEARACHGAIFGQLHDALDKYAEVK

SEQ ID NO: 33 - protein, 280 aa, Epichloe aotearoae
MATPTSTSTPIIFYDIAQRPPVAETCCAVNPWKSRLALNFKAVPYTTTWVKMPDISSVRASLNVPACRKFADGSDFNTLPIMHDPATSSLIGDSFDIAAYLQRTYPASGAGDLFPSQKLDYAVARDTQLLIPLSEIRASSELADYARFNSNVDAAFTAHVGLMVHGLPLDPATADVTKAEFVRRAGVSSWEDFEMVGEAREKMMQSLRNMLGDLAALFRRDASGPFLLGQRATYADLIVGGWLRMMRATLPASEWQEARACHGAIFGQLHDALDKYAEVK

SEQ ID NO: 34 - protein, 275 aa, Epichloe glyceriae
MATSTPIIFYDIAQRPPVAETCCAVNPWKSRLALNFKAVPYTTTWVSMPDISSVRASLNVPACRKFADGSDFNTLPIIHDPATGSLIGDSFDIAAHLQRAYPASGAGDLFPPQELDYVVARDTRLLVPLSETRASEFADYARFNSNVDAAFTAHVGLMVHGLPLDPATADVTKAEFVRRAGVSSWEDFELVGEAREKMMQSLRNVLGDLAALFRRDASGPFLLGQKATYADLIVGGWLRMMRATLPASEWQEARAWHGAVFGQLHDALDKYAEVK

SEQ ID NO: 35 - protein, 280 aa, Epichloe brachyelytri
MATSTSTSTPIIFYDIAQRPPVTETCCAVNPWKSRLALNFKAVPYTTTWVKMPDISSVRASLNVPACRKFADGSDFNTLPIIHDPATDSLIGDSFDIAAYLQRTYPASGAGDLFPPQKLDYAVSRDMQLLIPLSEMRASSELADYARFNSNVDAAFTAHVGLMVHGLPLDPATADVTKAEFVRRAGVSSWEDFEMVGEAREKMMQSLRNMLGDLAALFRRDASGPFLLGQKATYADLIVGGWLRMMRATLPASEWQEVRAWHGAIFGQLHDALDKYAEVK

SEQ ID NO: 36 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 37 - DNA, 846 bp, Thinopyrum elongatum
atggccacctccacctccacctccaccccaatcatcttctacgacatagcccagcggccccccgtcgcagaaacatgctgcgccgtcaacccttggaaatccagactggccctcaacttcaaggccgtcccctacacaaccacctgggtcaagatgccagatatcagcagcgtccgcgccagcctcaacgtgccagcgtgtcgcaagttcgccgacggctccgacttcaacaccctgcccatcatccacgaccccgcgaccgactccctcatcggcgactccttggacatcgccgcctacctgcagcgcacgtatcccgcctcgggcgccggcgacctcttccccccccagaagctcgactacgcagtcggcagggacatgcagcagctgctcttcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgctttgtttcggaaagatgcgagcgggccgttcctgttggggcagagggccacgtatgcggacatgattgtcggtggctggttgcgcatgatgcgggcgacgttgccggtgagcgagtggcaggaggcgagagcctgccacggggctatctttgggcagctgcatgatgcgctggacaagtatgccgaggtgaagtag

SEQ ID NO: 38 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgtctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 39 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatctcgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 40 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgtcgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 41 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccatctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 42 - DNA, 846 bp, Thinopyrum ponticum
atggccacctccgcctccacctccaccccaatcatcttctacgacatagcccagcggccccccgtcgcagaaacatgctgcgccgtcaacccttggaaattcagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 43 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctctgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 44 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctgattgcgcatgatgcgggcgacgttgccggtgagtgagtggcaggaggcgagagcctgccacggagctatctttgggcagctgcatgatgcgctggacaagtatgccgaggtgaagtag

SEQ ID NO: 45 - DNA, 846 bp, Thinopyrum ponticum
atggccacctccgcctccacctccaccccaatcatcttctacgacatagcccagcggccccccgtcgcaaaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 46 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatctcgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 47 - DNA, 846 bp, Thinopyrum ponticum
atggccacctccgcctccacctccaccccaatcatcttctacgacatagcccagcggccccccgtcgcagaaacatgctgcgccgtcaacccttggaaatccaaactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 48 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccttcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 49 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcactagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 50 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcacgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 51 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacacagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 52 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcttcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 53 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgatcaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 54 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgtcgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 55 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgtctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 56 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgtagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 57 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgtcggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 58 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcgcgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgatgttgccggtgagtgagtggcaggaggcgagagcctgccacggagctatctttgggcagctgcatgatgcgctggacaagtatgccgaggtgaagtag

SEQ ID NO: 59 - DNA, 846 bp, Thinopyrum ponticum
atggccacctccgcctccacctccaccccaatcatcttctacgacatagcccagcggccccccgtcgcagaaacatgctgcgccgtcaacccttggaaatccagactggccctcaacttcaaggccgtcccctacacaaccacctgggtgaagatgccagacatcagcagcgtccgcgccagcctcaacgtgccagcgtgtcgcaagttcgccgacggctccgacttcaacaccctgcccatcatccacgaccccgcgaccgactccctcgtcggcgactcctttgacatcgccgcctacctgcagcgcacgtatcccgcctcgggcgccggcgacctcttccccccccagaagctcgactacgcagtcggcagggacatgccgcagctgctcatcccgctgtccgagattcgcgcatcaccagagctcgcagactacgcccgcttcaacagcaacgttgacgcagcctttaccgcgcacgtgggcctcatggtccacggacttcccttggatcctgccaccgccgacgtgaccaaggccgagtttgtgcggcacgcggggctctcatcgtgggacgacttggaaatggttggcgaggcgcgcgacaagatgatgcagtccctccgaaacatgctgggggacctggctgccttgtttcggaaagatgcgagcgggccgttcctgttggggcagagggccacgtatgcggacatgattgtcggtggctggttgcgcatgatgcgggcgacgttgccggtgagtgagtggcaggaggcgagagcctgccacggagctatctttgggcagctgcatgatgcgctggacaagtatgccgaggtgaagtag

SEQ ID NO: 60 - DNA, 843 bp, Epichloe bromicola
atggccacctccacctccatctccaccccaatcatcttctacgacatagcccagcggccccccgtgacagaaacatgctgcgccgtcaacccttggaaatccagactggccctcaacttcaaggccgtcccctacacaaccacctgggtcaagatgccagacatcagcagcgtccgcgccagcctcaacgtgccagcgtgtcgcaaattcgccgacggctccgacttcaacaccctgcccatcatccacgaccccgcgaccgactccctcgtcggcgactccttcgacatcgccgcctacctgcagcgcacgtatcccgcctccggcgccggcgacctctttcccccccagaagctcgactacgcagtcggcagggacatgcagctgctcatcccgctgtccgagactcgggcatccccagagcttgcagactacgcccgcttcaacagcaacgttgacgcagcctttaccgcacacgtgggcctcatggtccacgggcttcccttggatcctgccaccgcggacgtgaccaaggccgagtttgtgcggcgcgcgggggtctcgtcgtgggaggacttcgaaatggttggtgaggcgcgcgagaagatgatgcagtccctccggaacatgctgggggacctggctgccttgttccggagagatgcgagcgggccgttcttgctgggacagaaggccacctatgcggatctgattgtcggtggctggctgcggatgatgcgggcgacgttgccggcgagtgagtggcaggaggtgagagcctgccacggggctgtcttcgggcaactgcatgatgcgctggacaagtatgccgaggtgaagtag

SEQ ID NO: 61 - DNA, 840 bp, Epichloe amarillans
atggccacctccacctccaccccaatcatcttctacgacatagcccagcggccccccgtcacagaaacgtgctgcgccgtcaacccttggaaatccagactggccctcaacttcaaggccgtcccctacacaaccacctgggtcaagatgccagacatcagcagcgtccgcgccagcctcaacctgccagcgtgtcgcaagttcgccgacggcaccgacttcgacaccctgcccatcatccacgaccccgcgaccggctccctcatcggcgactccttcgacatcgccgcctacctgcagcgcacgtatcccgcctccggcgccggcgacctcttccccccccagaagctcgactacgccgccggcagggacacgcagctgctcatcccgctgtccgaggttcgcgccgcatccccggagctcgcagactacgcccgcttcaacagcaacgttgacgcagccttcaccgcgcacgtgggcctcatggtccacgggcttcccttggaccctgccaccgcggacgtgaccaaggccgagtttgtgcggcgcgcggccgtctcatcgtgggacgacctcgacatggttggcgacgcgcgcgacaagatgatgcagtccctccggaacacgctgggggacctggccgccttgtttcggagagatgcgagcgggccgttcttgctgggacccaaggccacgtacgcggatctgattgtcggtggctggttgcgcatgatgcgggcgacgttgccgccgagtgagtggcaggcggcgagagcttggcacggggctgtcttcgggcagctgcatgatgcgctggacaagtacgccgaggtgaagtag

SEQ ID NO: 62 - DNA, 795 bp, Epichloe baconii
atggccacctccacctccacctccacctccacctccaccccaatcatcttctacgacatagcccagcggccccccgtcacagaaacatgctgcgccgtcaacccttggaaatccagactggccctcaacttcaaggccgtcccctacacaaccacctgggtcaagatgcccgacatcagcagcgtccgcgccagcctcaacctgccagcgtgtcgcaagttcgccgacggcaccgacttcaacaccctgcccatcatccacgaccccgcgaccggctccctcgtcggcgactccttcgacatcgccgcctacctgcagcgcacggacacgcagctgctcatcccgctgtccgaggttcgcgccgcatcctcggacctcgcagactacgcccgcttcaacagcaacgttgacgcagcctttaccgcgcacgtgggcctcatggtccacgggcttcccttggaccctgccaccgcggacgtgaccaaggccgagtttgtgcggcgcgcgggggtctcatcgtgggacgacttcgagatggcaggcgaggcgcgcgagaagatgatgcagtccctccggaacacgctgggggacctggccgccttgtttcggagagatgcgagcgggccgttcttgctgggacgcaaggccacgtacgcggatctgattgtcggtggctggttgcgcatgatgcgggcgacgttgccggcgagtgagtggcaggcggcgagagcttggcacggggctgtcttcgggcagctgcatgatgcgctggacaagtatgccgaggtgaagtag

SEQ ID NO: 63 - DNA, 861 bp, Epichloe festucae
atggccacctccacctccacctccacctccaccccaatcatcttctacgacatagcccagcggccccccgtcacagaaacatgctgcgccgtcaacccttggaaaaccagactggccctcaacttcaaggccgtcacctacacaaccacctgggtcaagatgccagacatcagcggcgtccgcgccagcctcaacgtgccagcgtgtcgcaaattcgccgacggcaccgacttcaacaccctgcccatcatccacgaccccgcgaccggctccctcatcggcgactccttcgacatcgccgcctacctgcagcgcacctatcccgcctccggcgctggccacctcttccccccccttcccccccctcagaagctcgactacgccgtcggcagggacatgcagctgctcatcccgctgtccgaggttcgcgcatcctcggagctcgcagactacgcccgcttcaacagcaacgttgacgcagcctttaccgcgcacgtcggcgtcatggtccacgggcttcccttggatcctgccaccgcggacgtgaccaaggccgagttcgtgcggcgcgcgggggtctcatcgtgggaggacttcgaaatggtcggtgaggcgcgcgagaagatgatgcagtccctccggaacatgctgggggacctggccgccttgtttcggagagatgcgagcgggccgttcttgctgggacagcaggccacgtacgcggatctgattgtcggtggctggttgcggatgatgcgggcgacgttgccggccagtgagtggcaggaggtgagagcttggcacggggctgtcttcgggcggctgcatgatgcgctggacaagtatgccgaggtgaagtag

SEQ ID NO: 64 - DNA, 846 bp, Epichloe gansuensis
atggccacctccacctccacttccgcctccaccccaatcatcttctacgacatagcccagcgcccccccgtcacagaaacatgctgcgccgtcaacccttggaaatccagactggccctcaatttcaaggccgtcccctacacaaccacctgggtcgagatgccagacatcagcagcgtccgcgccagtctcaacctgccagcgtgtcgcaaattcgccgacggctccgacttcaataccctgcccatcatccacgaccccgcgaccggctccctcatcggcgactccttcgacatcgccgcctacctgcagcgcacgtatcccgcctcgggcgccgacgacctcttccccccccagaagctggactacgtagtcggcagccatgtccagccgttcatcccgctgtctgacattcgcgcatcagagtttgcagattacgcccgcttcaacagcaacgttgacgcagcctttaccgcacacgtgggcctcatgctccacggacttcccttggatcctgccaccgcggacgtgaccaaggcagaattcgtgcgacgcgccggggtctcgtcgtgggaggattttgaaatggttggtgaggcgcgggagaagatgatgcagtcctttcggactatgctggaggacctggctgccttgttccggagagatgcgaccgggccgttcttgctgggacagaaggctacgtatgcggatctgattgtcggcgggtggttgcggatgatgcgcgcgacgttgccggcgagtgagtggcaggaggcgagagcttggcatggggccgtcttcggacaactgcatgatgcgctggacaagtatgccgaggtgaagtag

SEQ ID NO: 65 - DNA, 846 bp, Epichloe typhina
atggccacctcctccacctccacctccaccccaatcatcttctacgacatagcccagcggccccccgtcgcagaaacatgctgcgccgtcaacccttggaaatccagactggccctcaacttcaaggccgtcccctacacaaccacctgggtcaagatgccagacatcagcagcgtccgcgccagcctcaacgtgccagcgtgtcgtaaattcgccgacggctccgacttcaacaccctgcccatcatgcacgaccccgcgaccgactccctcatcggcgactccttcgatatcgccgcctacctgcagcgcacgtatcccgcctccggcgccggcgacctcttccccccccagaagctcgactacgcagtcggcagggacatgcagctgctcatcccgctgtccgaggtccgcgcatcaccagagctcgcagactacgcccgcttcaacagcaacgttgacgcagcctttaccgcgcacgtgggcctcatggtccacgggcttcccttggatcctgccaccgcagacgtgaccaaggccgagtttgtgcggcgcgcgggggtctcgtcgtgggaggacttcgaaatggttggcgaggtgcgcgagaagatgatgcagtccctccggaacatgctcggggacctggctgccttgtttcggagagatgcgagcgggccgttcctgctggggcagagggccacgtatgcggacctgattgtcggtggctggttgcgcatgatgcgcgcgacgttgccggcgagtgagtggcaggaggcgagagcctgccacggggccatcttcgggcagctgcatgatgcgctggacaagtatgccgaggtgaagtag

SEQ ID NO: 66 - DNA, 846 bp, Epichloe uncinata
atggccacctcctccacctccacctccaccccaatcatcttctacgacatagcccagcggccccccgtcgcagaaacatgctgcgccgtcaacccttggaaatccagactggccctcaacttcaaggccgtcccctacacaaccacctgggtcaagatgccagacatcagcagcgtccgcgccagcctcaacgtgccagcgtgtcgtaaattcgccgacggctccgacttcaacaccctgcccatcatgcacgaccccgcgaccgactccctcatcggcgactccttcgacatcgccgcctacctgcagcgcacgtatcccgcctccggcgccggcgacctcttccccccccagaagctcgactacgcagtcggcagggacatgcagctgctcatcccgctgtccgaggtccgcgcatcaccagagctcgcagactacgcccgcttcaacagcaacgttgacgcagcctttaccgcgcacgtgggcctcatggtccacgggcttcccttggatcctgccaccgcggacgtgaccaaggccgagtttgtgcggcgcgcgggggtctcgtcgtgggaggacttcgaaatggttggcgaggtgcgcgagaagatgatgcagtccctccggaacatgctcggggacctggctgccttgtttcggagagatgcgagcgggccgttcctgctggggcagagggccacgtatgcggacctgattgtcggtggctggttgcgcatgatgcgcgcgacgttgccggcgagtgagtggcaggaggcgagagcctgccacggggccatcttcgggcagctgcatgatgcgctggacaagtatgccgaggtgaagtag

SEQ ID NO: 67 - DNA, 843 bp, Epichloe sylvatica
atgaccacctccacctccacctccaccccaatcatcttctacgacatagcccagcggccccccgtcgcagaaacatgctgcgccgtcaacccttggaaatccagactggccctcaacttcaaggccgtcccctacaaaaccacctgggtcaagatgccagacatcagcagcgtccgcgccagcctcaaggtgccagcgtgtcgtaaattcgccgacggctccgacttcaacaccctgcccatcatgcacgaccccgcgaccgactccctcctcggcgactccttcgacatcgccgcctacctgcagcgcacgtatcccgcctccggcgccggcgacctcttccccccccagaagctcgactacgcagtcggcagggacatgcagctgctcatcccgctgtccgaggtccgcgcgtcaccagagctcgcagactacgcccgcttcaacagcaacgttgacgcagcctttaccgcgcacgtgggcctcatggtccacgggcttcccttggatcctgccaccgcggacgtgaccaaggccgagtttgtgcggcgcgcgggggtctcgtcgtgggaggaccttgaaatggttggcgaggcgcgcgagaagatgatgcagtccctccggaacatgctcggggacctggctgccttgtttcggagagatgcgagcgggccgttcctgctggggcagagggccacgtatgcggacctgattgtcggtggctggttgcgcatgatgcgcgcgacgttgccggcgagtgagtggcaggaggcgagagcctgccacggggccatcttcgggcagctgcatgatgcgctggacaagtatgccgaggtgaagtag

SEQ ID NO: 68 - DNA, 843 bp, Epichloe aotearoae
atggccacccccacctccacctccaccccaatcatcttctacgacatagcccagcggccccccgtcgcagaaacatgctgcgccgtcaacccttggaaatccagactggccctcaacttcaaggccgtcccctacacaaccacctgggtcaagatgccagacatcagcagcgtccgcgccagcctcaacgtgccagcgtgtcgcaagttcgccgacggctccgacttcaacaccctgcccatcatgcacgaccccgcgacctcttccctcatcggcgactccttcgacatcgccgcctacctgcagcgcacgtatcccgcctcgggcgccggcgacctcttcccctcccagaagctcgactacgcagtcgccagggacacgcagctgctcatcccgctgtccgagattcgcgcatcatcagagctcgcagactacgcccgcttcaacagcaacgttgacgcagcctttaccgcgcacgtgggcctcatggtccacgggcttcccttggatcctgccaccgccgacgtgaccaaggccgagtttgtgcggcgcgcgggcgtctcatcgtgggaggacttcgaaatggttggcgaggcgcgcgagaagatgatgcagtccctccggaacatgctgggggacctggctgccttgtttcggagagatgcgagcgggccgttcctgctggggcagagggccacgtatgcggacctgattgtcggtggctggttgcgcatgatgcgggcgacgttgccggcgagtgagtggcaggaggcgagagcctgccacggggctatcttcgggcagctgcatgatgcgctggacaagtatgccgaggtgaagtag

SEQ ID NO: 69 - DNA, 828 bp, Epichloe glyceriae
atggccacctccaccccaatcatcttctacgacatagcccagcggccccccgtcgcagaaacatgctgcgccgtcaacccttggaaatccagactggccctcaacttcaaggccgtcccctacacaaccacctgggtcagcatgccagacatcagcagcgtccgcgccagcctcaacgtgccggcgtgtcgcaaattcgccgacggctccgacttcaacaccctgcccatcatccacgaccccgcgaccggctccctcatcggcgactccttcgacatcgccgcccacctgcagcgcgcctatcccgcctccggcgccggcgacctcttccccccccaggagctggactacgtggtcgccagggacacgcggctgctcgtcccgctgtccgagactcgcgcatcagagttcgcggactacgcccgcttcaacagcaacgttgacgcagcctttaccgcacacgtgggcctcatggtccacgggcttcccttggaccctgccaccgcggacgtgaccaaggcggagtttgtgcggcgcgcgggagtctcgtcgtgggaggatttcgaattggttggtgaggcgcgcgagaagatgatgcagtccctccggaacgtgctgggggacctggctgccttgtttcggagagatgcgagcgggccgttcttgctgggacagaaggccacgtatgcggatctgattgtcggtggctggttgcggatgatgcgggcgacgttgcccgcgagtgagtggcaggaggcgagagcctggcatggcgctgtcttcgggcagctgcatgatgcgctggacaagtatgccgaggtgaagtag

SEQ ID NO: 70 - DNA, 843 bp, Epichloe brachyelytri
atggccacctccacctccacctccaccccaatcatcttctacgacatagcccagcggccccccgtcacagaaacatgctgcgccgtcaacccttggaaatccagactggccctcaacttcaaggccgtcccctacacaaccacctgggtcaagatgccagacatcagcagcgtccgcgccagcctcaacgtgcctgcgtgtcgcaaattcgccgacggctccgacttcaacaccctgcccatcatccacgaccccgcgaccgactccctcatcggcgactccttcgacatcgccgcctacctgcagcgcacgtatcccgcctccggcgccggcgacctcttccccccccagaagctcgactacgcagtcagcagggacatgcagctgctcatcccgctgtccgagatgcgcgcatcatcagagctcgcagactacgcccgcttcaacagcaacgttgacgcagcctttaccgcgcacgtgggcctcatggtccacgggcttcccttggatcctgccaccgcggacgtgaccaaggccgagtttgtgcggcgcgcgggggtctcatcgtgggaggatttcgaaatggttggtgaggcgcgcgagaagatgatgcagtccctccggaacatgctgggggacctggctgccttgtttcggagagatgcgagcgggccgttcttgctggggcagaaggccacgtatgcggatctgattgtcggtggctggttgcgcatgatgcgggcgacgttgccggcgagtgagtggcaggaggtgagagcctggcacggggctatcttcgggcagctgcatgatgcgctggacaagtatgccgaggtgaagtag

(19) United States
(12) Patent Application Publication
(10) Pub. No.: US 2023/0151371 A1
(43) Pub. Date: May 18, 2023

(54) POLYPEPTIDES HAVING EPOXY GROUP-REMOVING CATALYTIC ACTIVITY, NUCLEIC ACIDS ENCODING THE POLYPEPTIDES AND USE THEREOF

(71) Applicant: SHANDONG VEZYME BIOTECH CO., LTD., Jinan City (CN)

(72) Inventor: Hongwei WANG, Jinan City (CN)

(21) Appl. No.: 17/905,586

(22) PCT Filed: Dec. 11, 2020
(86) PCT No.: PCT/CN2020/135822

§ 371 Date: Sep. 2, 2022

(30) Foreign Application Priority Data
Mar. 5, 2020 (CN) 202010147965.9
Mar. 5, 2020 (CN) 202010147974.8
Mar. 5, 2020 (CN) 202010148668.6
Jun. 9, 2020 (CN) 202010520261.1

(51) Int. Cl. C12N 15/52; A23L 5/20; A61K 38/16; A61P 39/02; C12N 15/74; C12Q 1/25; C12N 9/00

(52) U.S. Cl. CPC C12N 15/52; A23L 5/20; A61K 38/16; A61P 39/02; C12N 15/74; C12Q 1/25; C12N 9/00

(57) ABSTRACT

A polypeptide having epoxy group-removing catalytic activity with an amino acid sequence as set forth in SEQ ID NOs: 1-35, a nucleic acid molecule encoding the polypeptide, a nucleic acid construct comprising the nucleic acid, a pharmaceutical composition for detoxification and a food, beverage or feed composition comprising the polypeptide, and a host cell and an engineered microorganism into which the nucleic acid is introduced. Disclosed are a method for producing the polypeptide; and a method for catalyzing a reaction of removing an epoxy group of a trichothecene, a method for preventing cell poisoning or relieving cytotoxicity, a method for processing a food and beverage or feed composition, and a method for reducing or decreasing a toxin in a composition, all using the polypeptide. Further disclosed are a glutathionylated derivative, a method for evaluating the detoxification effect for a sample contaminated with a trichothecene using the glutathionylated derivative.

### RELATED APPLICATIONS

The present application is a U.S. National Phase of International Application Number PCT/CN2020/135822 filed Dec. 11, 2020, and claims priority to Chinese Application Numbers CN202010147965.9 filed Mar. 5, 2020, CN202010147974.8 filed Mar. 5, 2020, CN202010148668.6 filed Mar. 5, 2020, and CN202010520261.1 filed Jun. 9, 2020.

### INCORPORATION BY REFERENCE

The sequence listing provided in the file entitled Sequence_Listing_C6392-010_v1.txt, which is an ASCII text file that was created on Sep. 2, 2022, and which comprises 124,649 bytes, is hereby incorporated by reference in its entirety.

### TECHNICAL FIELD

The present invention relates to the field of polypeptides, in particular to a polypeptide having epoxy group-removing catalytic activity, a nucleic acid encoding the polypeptide and use thereof.

### BACKGROUND ART

Trichothecene mycotoxins have a basic chemical structure of sesquiterpene, and are also called 12,13-epoxytrichothecenes as an epoxy group is formed between the 12-position carbon and the 13-position carbon. Since the 1970s, scientific researchers have proved that the epoxy group of trichothecene mycotoxins is the main group as the source of toxicity. At present, trichothecene mycotoxin derivatives with the epoxy group removed can be chemically synthesized in vitro under highly alkaline conditions; however, this process is hardly applied in industry due to harsh reaction conditions and low efficiency. In addition, several anaerobic bacteria have been isolated from animal gut microbes that can detoxify trichothecene mycotoxins; however, the mechanism of action is unclear, and practical industrial applications have been greatly limited due to the dependence on anaerobic conditions.

### SUMMARY OF THE INVENTION

In view of the problems existing in the prior art, the inventor provides a polypeptide having epoxy group-removing catalytic activity, which is capable of catalyzing a reaction between an epoxy group of a trichothecene mycotoxin and glutathione (abbreviated as GSH) under mild conditions to produce a non-toxic and harmless glutathionylated derivative, thereby achieving detoxification of the trichothecene mycotoxin. The present invention has been accomplished based at least in part on this, and specifically, the present invention comprises the following contents.

A first aspect of the present invention provides an isolated polypeptide having epoxy group-removing catalytic activity, which is capable of catalyzing a reaction between a trichothecene mycotoxin and GSH in a PBS buffer at a temperature of 15° C. to 35° C. to remove an epoxy group and produce a glutathionylated derivative. Here, although the reaction temperature of 15° C. to 35° C. is defined, it is only to characterize or identify that the polypeptide has epoxy group-removing catalytic activity under this condition, and it does not mean that the polypeptide of the present invention does not have epoxy group-removing catalytic activity at a temperature below 15° C. or above 35° C. In fact, the conditions for the catalytic reaction of the active polypeptide of the present invention are not limited to the above-mentioned temperatures.

A second aspect of the present invention provides an isolated polypeptide having epoxy group-removing catalytic activity, comprising an amino acid sequence selected from the group consisting of the following (1) to (5):

(1) An amino acid sequence as set forth in any of SEQ ID Nos: 1-35, wherein SEQ ID NO: 1 represents an amino acid sequence derived from Thinopyrum ponticum, SEQ ID NO: 2 represents an amino acid sequence derived from Thinopyrum elongatum, SEQ ID Nos: 3-24 represent mutant sequences of SEQ ID NO: 1 that have been verified to have the original activity, and SEQ ID Nos: 25-35 represent amino acid sequences derived from different species of Epichloë.

(2) An amino acid sequence which has 85% or more, preferably 95% or more, more preferably 97% or more, still preferably 98% or more, further preferably 99% or more sequence identity with the amino acid sequence of (1), and is derived from the same genus, preferably the same species; and it is further preferred that the polypeptides composed of these sequences still have the original enzyme activity. In certain embodiments, the sequence identity of the amino acid sequence of the active polypeptide and the amino acid sequence of (1) is 95% or more, and all these sequences are derived from Epichloë.

(3) An amino acid sequence which has one or more amino acid mutations and has 85% or more, preferably 90% or more, still preferably 95% or more, more preferably 97% or more, still preferably 98% or more, further preferably 99% or more sequence identity as compared with the amino acid sequence of (1) or (2), and still maintains the original protein activity. The amino acid mutation herein comprises insertions, deletions or substitutions of amino acids.

(4) A partial consecutive sequence derived from the amino acid sequence of any of (1) to (3), preferably, the polypeptide (or truncated polypeptide) having the partial consecutive sequence still has the original enzymatic catalytic activity of the polypeptide, and more preferably, it has a partial consecutive sequence located at the N-terminal of the amino acid sequence of any of (1) to (3), for example, a polypeptide having the first 200 to 250 amino acids from the N-terminal, e.g., a polypeptide having the first 208 amino acids from the N-terminal, or a polypeptide having the first 242 amino acids from the N-terminal.

(5) A chimeric sequence in which an additional amino acid sequence is linked to the N-terminal and/or C-terminal of the amino acid sequence of any of (1) to (4). That is, the active polypeptide of the present invention may be a chimeric polypeptide. In certain embodiments, the additional amino acid sequences are sequences that enhance expression or secretion of the polypeptide, examples of which include, but are not limited to, leader peptides, signal peptides, and transit peptides. In certain embodiments, the active polypeptide is a chimeric polypeptide of an active fragment of a full-length protein and an additional amino acid sequence, wherein the additional amino acid sequence is a sequence corresponding to an additional homologous protein other than the active fragment, e.g., a sequence of a structural region or a functional region. For example, when the full length of an enzyme derived from a species is composed of two portions, A and B, the full length of an additional homologous enzyme of the same genus but of a different species is composed of two portions, A′ and B′, and A and A′ are homologous corresponding regions and B and B′ are homologous corresponding regions, the chimeric polypeptide may be composed of A′+B or A+B′. In certain embodiments, the additional amino acid sequence comprises a non-functional sequence, e.g., a linker arm or a spacer sequence. In certain embodiments, the additional amino acid sequence is independently functional polypeptides linked to the active polypeptide of the present invention via a non-functional sequence, e.g., a linker arm or a spacer sequence.

In certain embodiments, the active polypeptide of the present invention has a conserved site selected from at least one of: amino acid A at position 98, and amino acid A at position 99.

A third aspect of the present invention provides an isolated active polypeptide (having epoxy group-removing catalytic activity), having an amino acid sequence of:

V1-GDX1X2DIAAX3LQRT-V2-ADYARFNX1NVDX4AFX5AHV
X1X6MX6HGLPLDPAX7X4DVX8KAEFVR-V3,

wherein:

X1 represents G or S; X2 represents F or L; X3 represents Y or H; X4 represents A or V; X5 represents T or Q or N; X6 represents L or V; X7 represents T or S; and X8 represents T or I;

V1 is absent or represents a first variable region, the amino acid sequence of the first variable region corresponds to a sequence of a plurality of consecutive amino acids before the amino acid at position 92 in SEQ ID NO: 1, and the sequence identity of the first variable region with the sequence of the plurality of consecutive amino acids is 80% or more, 85% or more, preferably 90% or more, preferably 92% or more, more preferably 95% or more, further preferably 98% or less, e.g., 99%;

V2 represents a linker arm or represents a second variable region, the amino acid sequence of the second variable region corresponds to a sequence of a plurality of consecutive amino acids between the amino acids at positions 105 to 143 in SEQ ID NO: 1, and the sequence identity of the second variable region with the sequence of the plurality of consecutive amino acids is 80% or more, 85% or more, preferably 90% or more, preferably 92% or more, more preferably 95% or more, further preferably 98% or less, e.g., 99%; and

V3 is absent or represents a third variable region, the amino acid sequence of the third variable region corresponds to a sequence of a plurality of consecutive amino acids after the amino acid at position 144 in SEQ ID NO: 1, and the sequence identity of the third variable region with the sequence of the plurality of consecutive amino acids is 80% or more, 85% or more, preferably 90% or more, preferably 92% or more, more preferably 95% or more, further preferably 98% or less, e.g., 99%.

A fourth aspect of the present invention provides an isolated nucleic acid molecule encoding the polypeptide according to the first aspect or the second aspect.

A fifth aspect of the present invention provides an isolated nucleic acid molecule having a base sequence selected from the group consisting of the following (a) to (e):

(a) A sequence as set forth in any of SEQ ID Nos: 36-70. SEQ ID NO: 36 represents the de-epoxidase gene derived from Thinopyrum ponticum, SEQ ID NO: 37 represents the de-epoxidase gene derived from Thinopyrum elongatum, SEQ ID Nos: 38-59 represent mutants of the sequence of SEQ ID NO: 36, and SEQ ID Nos: 60-70 represent homologous gene sequences derived from different species of Epichloë.

(b) A sequence modified for the host codon bias based on the base sequence of (a). In order to adapt to the needs of different hosts, the base sequence of (a) can be modified for the codon bias according to codon degeneracy. The modification for the codon bias generally does not change the sequence of the product protein or polypeptide.

(c) A conserved region sequence of the sequence as set forth in (a). A conserved region sequence encoding an active polypeptide is preferred. It should be noted that the conserved region sequence of bases does not necessarily express or encode an active polypeptide. As long as it is a conserved region, it can be used as a detection target.

(d) A sequence which has 85% or more, preferably 90% or more, still preferably 95% or more, still preferably 97% or more, more preferably 98% or more, most preferably 99% or more sequence identity with any of (a) to (c), and is derived from the same genus, preferably the same species.

(e) A sequence complementary to at least a portion of any of the sequences of (a) to (d). The complementary sequence comprises a sequence that specifically hybridizes to these sequences under stringent conditions, for example, a probe, a primer, and the like.

A sixth aspect of the present invention provides a nucleic acid construct, comprising the nucleic acid according to the fourth and fifth aspects of the present invention and optionally a regulatory element. Examples of regulatory elements include, but are not limited to, a promoter, an activator, an enhancer, an operon, a ribosome binding site, a start signal, a stop signal, a cap signal, a polyadenylation signal, and other signals involved in transcriptional or translational control, and the like. These regulatory elements enable expression of a nucleic acid molecule in an intended target cell (e.g., Escherichia coli, yeast cells, and the like). A nucleic acid construct comprises a self-replicating construct, and also comprises a non-self-replicating construct. Examples of self-replicating constructs include, but are not limited to, vectors, plasmids, and the like.

A seventh aspect of the present invention provides a pharmaceutical composition for detoxification, comprising a polypeptide having epoxy group-removing activity or a nucleic acid encoding therefor and optionally a pharmaceutically acceptable carrier, wherein the active polypeptide is capable of catalyzing a reaction between a trichothecene mycotoxin and glutathione to produce a glutathionylated derivative, thereby removing epoxy groups that cause toxin toxicity. The pharmaceutical composition for detoxification comprises a cell or a cell component. The cell here refers to an in vitro cell that can be administered to a human body. The cell comprises or is capable of expressing the active polypeptide of the present invention. In the present invention, the pharmaceutically acceptable carrier is a carrier well known in the art, and one of ordinary skill in the art can determine that it meets clinical standards. The pharmaceutically acceptable carrier includes, but is not limited to, a diluent and an excipient. The pharmaceutical composition for detoxification of the present invention may be in any suitable dosage form, for example, an injection, a suspension, an emulsion, and the like. It can be administered into the body by known means. For example, it can be delivered into a tissue of interest by intramuscular injection, optionally administered via intravenous, transdermal, intranasal, oral, mucosal, or other delivery means. Such administration may be via single or multiple doses. It is understood by those skilled in the art that the actual dosage to be administered herein may vary greatly depending on a variety of factors, such as target cells, the organism type or the tissue, the general condition of the subject to be treated, the route of administration, the mode of administration, and the like.

An eighth aspect of the present invention provides a food and beverage or feed composition, comprising de-epoxidase which is capable of catalyzing a reaction between a trichothecene mycotoxin and glutathione in a PBS buffer at a temperature of 15° C. to 35° C. to produce a glutathionylated derivative. Here, although the reaction temperature of 15° C. to 35° C. is defined, it is only to characterize or identify that the polypeptide has epoxy group-removing catalytic activity under this condition, and it does not mean that the active polypeptide of the present invention does not have epoxy group-removing catalytic activity at a temperature below 15° C. or above 35° C. In fact, the conditions for the catalytic reaction of the active polypeptide of the present invention are not limited to the above-mentioned temperatures.

A ninth aspect of the present invention provides a host cell, comprising the nucleic acid according to the fourth and fifth aspects of the present invention introduced by means of genetic engineering, or the nucleic acid construct according to the sixth aspect of the present invention. The host cell is not particularly limited, and comprises a prokaryotic cell and a eukaryotic cell. Examples of prokaryotic cells include, but are not limited to, Escherichia coli, and the like, and examples of eukaryotic cells include, but are not limited to, a yeast cell, a plant cell or an animal cell.

A tenth aspect of the present invention provides an engineered microorganism, comprising an exogenously introduced gene derived from Thinopyrum and/or Epichloë, and the gene has the nucleic acid base sequence according to the fifth aspect of the present invention. The present invention further provides a feed additive, a biological fertilizer or a biological pesticide, comprising the engineered microorganism according to the tenth aspect, and in this case, the engineered microorganism is a dry powder.

An eleventh aspect of the present invention provides a method for producing an active polypeptide. The production method of the present invention comprises a genetic engineering method and a chemical synthesis method. The genetic engineering method comprises allowing the nucleic acid of the present invention to be expressed in an intracellular (e.g., Escherichia coli) or non-cellular expression system, thereby obtaining a polypeptide. The chemical synthesis method may use any method currently known.

A twelfth aspect of the present invention provides a method for catalyzing a reaction of removing an epoxy group of a trichothecene, comprising contacting the active polypeptide according to the first and second aspects of the present invention, or the host cell according to the ninth aspect with a trichothecene and GSH under conditions suitable for the reaction, thereby producing a glutathionylated derivative. The conditions suitable for the reaction in the present invention comprise a reaction temperature of 1° C. to 45° C., preferably 2° C. to 40° C., more preferably 5° C. to 35° C., further preferably 10° C. to 30° C.; a reaction time of 10 minutes to 36 hours, e.g., 10 to 60 minutes, and 1.5 to 24 hours; and an appropriate reaction solution, e.g., a PBS solution or a DMSO solution, with a pH between 4.0 and 7.5, preferably between 4.5 and 7.0. The specific reaction conditions need to be adjusted by those skilled in the art as needed according to the source of the enzyme, the enzyme activity, concentrations of substrates, the amount of reaction and the like, and are not particularly limited.

A thirteenth aspect of the present invention provides a method for preventing cell poisoning or relieving cytotoxicity, comprising contacting a cell to be treated with a polypeptide having epoxy group-removing activity or a nucleic acid encoding therefor, or a cell producing the active polypeptide, and optionally glutathione. The cell to be treated in the present invention is an in vitro cell, e.g., an animal cell. The cell producing the polypeptide having epoxy group-removing activity comprises a yeast cell, Escherichia coli, and the like.

A fourteenth aspect of the present invention provides a method for processing a food and beverage or feed composition, comprising contacting a food and beverage or feed raw material with de-epoxidase or a cell producing the enzyme under conditions suitable for the reaction. The cell producing the enzyme may be, for example, a host cell into which a nucleic acid molecule capable of producing the enzyme according to the first aspect is introduced by means of genetic engineering, and the cell is contacted with a trichothecene and GSH, thereby producing a glutathionylated derivative. Such host cell may be, for example, a prokaryotic cell or a eukaryotic cell. Examples of prokaryotic cells include, but are not limited to, Escherichia coli, and the like, and examples of eukaryotic cells include, but are not limited to, a yeast cell, a plant cell or an animal cell. The specific reaction conditions of the method for processing a food and beverage or feed composition of the present invention need to be adjusted by those skilled in the art as needed according to the source of the enzyme, the enzyme activity, concentrations of substrates, the amount of reaction and the like, and are not particularly limited.

A fifteenth aspect of the present invention provides a method for reducing or decreasing a toxin in a composition, comprising contacting a food and beverage or feed raw material comprising a toxin with de-epoxidase or a cell producing the enzyme under conditions suitable for the reaction, wherein the toxin is a trichothecene.

A sixteenth aspect of the present invention provides a glutathionylated derivative, having a structure shown in the following general formula (I):

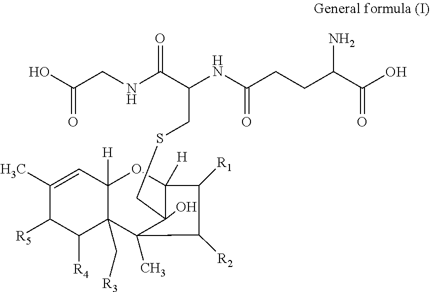

wherein each of R₁, R₂ and R₃ independently represents a hydrogen atom, a hydroxyl group or an ester group represented by —OCO—R′, wherein R′ is a linear or branched C₁-C₅ alkyl group, R₄ represents a hydrogen atom or a hydroxyl group, and R₅ represents a hydrogen atom, ═O, a hydroxyl group or an ester group represented by —OCO—R″, wherein R″ is a linear or branched C₁-C₁₀ alkyl group.

A seventeenth aspect of the present invention provides the use of the glutathionylated derivative of the present invention as an index for evaluating a reaction of removing an epoxy group of a trichothecene.

An eighteenth aspect of the present invention provides a method for evaluating the detoxification effect for a sample contaminated with a trichothecene, comprising using the glutathionylated derivative of the present invention as an evaluation index.

A nineteenth aspect of the present invention provides a method for evaluating the detoxification effect for a sample contaminated with a trichothecene, comprising:

(1) measuring the content of the glutathionylated derivative in the sample to obtain a measured value, or measuring a ratio of the content of the glutathionylated derivative to the content of the trichothecene in the sample;

(2) comparing the measured value or the ratio with a reference value; and

(3) evaluating the detoxification effect for the sample according to the comparison result.

In certain embodiments, the reference value here is a result obtained from a control sample, or the content of the glutathionylated derivative in the sample before treatment, or a ratio of the content of the glutathionylated derivative to the content of the trichothecene.

A twentieth aspect of the present invention provides a method for determining the epoxy group-removing catalytic activity of a polypeptide, comprising treating a standard sample with the polypeptide, and measuring the content of a glutathionylated derivative of the present invention, or the content of a trichothecene, or a ratio of the content of the glutathionylated derivative to the content of the trichothecene in the standard sample before and after treatment. The standard sample is a standard sample of a trichothecene. The ratio of the content of the glutathionylated derivative to the content of the trichothecene comprises the content of the glutathionylated derivative: the content of the trichothecene, and further comprises the content of the trichothecene: the content of the glutathionylated derivative.

A twenty-first aspect of the present invention provides a method for identifying a compound capable of affecting the epoxy group-removing catalytic activity of a polypeptide, comprising:

a. contacting the polypeptide with a standard sample of a trichothecene under conditions suitable for the reaction to obtain a reaction system, and measuring the first production rate of a glutathionylated derivative;

b. applying a compound to be tested to the same reaction system as step a, and measuring the second production rate of a glutathionylated derivative; the same reaction system as step a comprises another reaction system of the same components and contents in the reaction mixture, and further comprises the situation of the same reaction system in different time periods; and

c. comparing the first production rate and the second production rate, and when the second production rate is less than the first production rate, identifying the compound to be tested as a polypeptide activity-inhibiting compound; when the second production rate is greater than the first production rate, identifying the compound to be tested as a polypeptide activity-promoting compound; and when the second production rate is equal to the first production rate, identifying the compound to be tested as a compound that is ineffective for the polypeptide activity.

A twenty-second aspect of the present invention provides the use of the active polypeptide of the present invention in food processing, feed processing and pharmaceutical manufacturing.

A twenty-third aspect of the present invention provides the use of the nucleic acid of the present invention in plant breeding and disease control.

### BRIEF DESCRIPTION OF THE DRAWINGS

FIG. 1 shows a graph of SDS-PAGE analysis after purification of FTCD.

FIGS. 2A and 2B show the effect of enzyme amount on the enzymatic reaction. Panel FIG. 1A shows the reduction of the enzymatic reaction substrate, vomitoxin (DON); and panel FIG. 2B shows the production of the enzymatic reaction product, DON-GSH.

FIGS. 3A and 3B show the effect of pH of the reaction buffer on the enzymatic reaction. Panel FIG. 3A shows the reduction of the enzymatic reaction substrate, vomitoxin (DON); and panel FIG. 3B shows the production of the enzymatic reaction product, DON-GSH.

FIGS. 4A and 4B show the effect of the reaction temperature on the enzymatic reaction. Panel FIG. 4A shows the reduction of the enzymatic reaction substrate, vomitoxin (DON); and panel FIG. 4B shows the production of the enzymatic reaction product, DON-GSH.

FIG. 5A shows extracted ion chromatograms (EICs) of in vitro enzymatic reaction of DON and GSH by LC-HRMS (Method 1).

FIG. 5B shows an LC-HRMS₂ (Method 2) mass spectrogram of the product ions produced by the high-energy collision induced dissociation of DON-GSH obtained by in vitro enzymatic reaction of DON and GSH.

FIG. 6A shows extracted ion chromatograms (EICs) of in vitro enzymatic reaction of 3-ADON and GSH by LC-HRMS (Method 1).

FIG. 6B shows an LC-HRMS₂ (Method 2) mass spectrogram of the product ions produced by the high-energy collision induced dissociation of 3-ADON-GSH obtained by in vitro enzymatic reaction of 3-ADON and GSH.

FIG. 7A shows extracted ion chromatograms (EICs) of in vitro enzymatic reaction of 15-ADON and GSH by LC-HRMS (Method 1).

FIG. 7B shows an LC-HRMS₂ (Method 2) mass spectrogram of the product ions produced by the high-energy collision induced dissociation of 15-ADON-GSH obtained by in vitro enzymatic reaction of 15-ADON and GSH.

FIG. 8A shows extracted ion chromatograms (EICs) of in vitro enzymatic reaction of NIV and GSH by LC-HRMS (Method 1).

FIG. 8B shows an LC-HRMS₂ (Method 2) mass spectrogram of the product ions produced by the high-energy collision induced dissociation of NIV-GSH obtained by in vitro enzymatic reaction of NIV and GSH.

FIG. 9A shows extracted ion chromatograms (EICs) of in vitro enzymatic reaction of Fus-X and GSH by LC-HRMS (Method 1).

FIG. 9B shows an LC-HRMS₂ (Method 2) mass spectrogram of the product ions produced by the high-energy collision induced dissociation of Fus-X-GSH obtained by in vitro enzymatic reaction of Fus-X and GSH.

FIG. 10A shows extracted ion chromatograms (EICs) of in vitro enzymatic reaction of DAS and GSH by LC-HRMS (Method 1).

FIG. 10B shows an LC-HRMS₂ (Method 2) mass spectrogram of the product ions produced by the high-energy collision induced dissociation of DAS-GSH obtained by in vitro enzymatic reaction of DAS and GSH.

FIG. 11A shows extracted ion chromatograms (EICs) of in vitro enzymatic reaction of HT-2 and GSH by LC-HRMS (Method 1).

FIG. 11B shows an LC-HRMS₂ (Method 2) mass spectrogram of the product ions produced by the high-energy collision induced dissociation of an HT-GSH adduct obtained by in vitro enzymatic reaction of HT-2 and GSH.

FIG. 12A shows extracted ion chromatograms (EICs) of in vitro enzymatic reaction of T-2 and GSH by LC-HRMS (Method 1).

FIG. 12B shows an LC-HRMS₂ (Method 2) mass spectrogram of the product ions produced by the high-energy collision induced dissociation of an T2-GSH adduct obtained by in vitro enzymatic reaction of T-2 and GSH.

FIG. 13 shows the effect of trichothecenes on the viability of human cell lines. OD450 nm was measured after cells were treated with different concentrations of DON(a), 3ADON(b), 15ADON(c), FUS-X(d), NIV(e), T-2(f), HT-2(g), and DAS(h) for 48 h.

FIG. 14 shows extracted ion chromatograms of toxin-treated transgenic yeast by LC-HRMS (Method 1).

FIG. 15 shows the DON tolerance results of FTCD transgenic Pichia pastoris.

FIG. 16 shows a phylogenetic tree of FTCD and its homologous sequences.

FIG. 17 shows extracted ion chromatograms of DON-treated FTCD homologous sequence transgenic yeast by LC-HRMS (Method 1). The DON-GSH adduct was detected in positive ion mode, with an m/z of 604.21730 (corresponding to [M+H]⁺, Δ±5 ppm).

FIG. 18A shows SDS-PAGE results: M represents protein markers, lane 1 represents protein expressed at 4 h, and lane 2 represents protein expressed at 8 h; and FIG. 18B lane 1 represents blank plasmid, and lane 2 represents the target gene.

FIGS. 19A-19E show the clearance of trichothecene mycotoxins in feed samples by various probiotics comprising FTCD. Panel FIG. 19A shows treatment of feed with Bacillus comprising FTCD; panel FIG. 19B shows treatment of feed with Lactobacillus comprising FTCD; panel FIG. 19C shows treatment of feed with Bifidobacterium comprising FTCD; panel FIG. 19D shows treatment of feed with Saccharomyces cerevisiae comprising FTCD; and panel FIG. 19E shows treatment of feed with Pichia pastoris comprising FTCD. Samples were taken at 0 h, 0.5 h, 1 h, and 2 h of treatment for LC-HRMS analysis, respectively. It was found that the relative contents of DON, 3-ADON, 15-ADON, NIV, T-2 and HT-2 toxins were significantly reduced by treatment with different probiotics comprising FTCD, but the detoxification capability of different strains was slightly different.

FIGS. 20A-20C show clearance results of DON in highly processed products of maize by FTCD protein purified in vitro.

FIGS. 21A and 21B show clearance results of DON in two brands of apple juice by FTCD protein purified in vitro.

### DETAILED DESCRIPTION OF EMBODIMENTS

Various exemplary implementations of the present invention are now described in detail. The detailed description should not be considered as a limitation on the present invention, but should be understood as a more detailed description of certain aspects, characteristics, and embodiments of the present invention. “%” is a percentage based on weight, unless otherwise specified.

Herein, the terms “polypeptide” and “protein” are used interchangeably and refer to a polymer of amino acid residues as well as variants and synthetic and naturally occurring analogs thereof. Both terms apply to an amino acid polymer in which one or more amino acid residues are synthetic, non-naturally occurring amino acids (such as chemical analogs of the corresponding naturally occurring amino acids), as well as to a naturally occurring amino acid polymer and a naturally occurring chemical derivative thereof. Such chemical derivatives comprise, for example, post-translational modification and degradation products, comprising pyroglutamylated, isoaspartylated, proteolytic, phosphorylated, glycosylated, oxidized, isomerized and deaminated variants.

Herein, the term “active polypeptide” refers to a polypeptide having catalytic activity of de-epoxidase, i.e., an active polypeptide that converts an epoxy group into another group or removes the epoxy group. It is also sometimes referred to herein as an “enzyme”.

Herein, the term “sequence identity” refers to the degree to which sequences are identical on a nucleotide-by-nucleotide basis or on an amino acid-by-amino acid basis within a comparison window. Thus, the percent sequence identity can be calculated by comparing the two optimally aligned sequences in a comparison window, determining the number of positions where the same nucleic acid base or the same amino acid residue occurs in the two sequences to obtain a number of matched positions, dividing the number of matched positions by the total number of positions in the comparison window (i.e., window size), and multiplying the result by 100 to obtain the percent sequence identity.

Herein, the calculation of sequence identity or sequence similarity (used interchangeably herein) between two sequences is performed by the following method. To determine the percent identity of two amino acid sequences or two nucleic acid sequences, the sequences are aligned for optimal comparison purposes (for example, gaps can be introduced in one or both of the first and second amino acid or nucleic acid sequences for optimal alignment, and non-homologous sequences can not be taken into consideration for comparison purposes). In certain embodiments, the length of the reference sequence aligned for comparison purposes is at least 30%, preferably at least 40%, more preferably at least 50%, still preferably at least 70%, 80% and 90%, even 100% of the entire length of the reference sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percent identity between two sequences is a function of the number of identical positions shared by the sequences, where the number of gaps and the length of each of gaps which needs to be introduced for optimal alignment of the two sequences are taken into account. Sequence comparison and determination of percent identity between two sequences can be accomplished using mathematical algorithms. The percent identity between two amino acid sequences or between two nucleotide sequences can be determined using the GAP program in the GCG software package (available at http://www.gcg.com) or the ALIGN program (Version 2.0).

Herein, for the term “amino acid at position x” or similar expressions, the amino acid sequence of the de-epoxidase derived from Thinopyrum ponticum is taken as a position reference, that is, the amino acid sequence as set forth in SEQ ID NO: 1 is used as a position reference, unless explicitly specified otherwise. Similarly, for the term “base at position y” or similar expressions, the base sequence of the de-epoxidase gene derived from Thinopyrum ponticum is taken as a position reference, that is, the base sequence as set forth in SEQ ID NO: 36 is used as a position reference, unless explicitly specified otherwise.

Herein, the term “trichothecene mycotoxin” or “trichothecene” refers to a generic term for a class of compounds which have a basic chemical structure of sesquiterpene, and in which an epoxy group is formed between the 12-position carbon and the 13-position carbon. Preferably, the trichothecene mycotoxin has a structure shown in the following general formula (II):

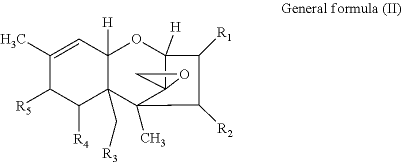

wherein each of R₁, R₂ and R₃ independently represents a hydrogen atom, a hydroxyl group or an ester group represented by —OCO—R′, wherein R′ is a linear or branched C₁-C₅ alkyl group, e.g., CH₃, CH₂CH₃, CH₂CH₂CH₃ or CH₂(CH₃)₂, R₄ represents a hydrogen atom or a hydroxyl group, and R₅ represents a hydrogen atom, ═O, a hydroxyl group or an ester group represented by —OCO—R″, wherein R″ is a linear or branched C₁-C₁₀ alkyl group, preferably CH₃ and CH₂CH₃, still preferably a linear or branched C₃-C₈ alkyl group, more preferably CH₂CH(CH₃)₂. In certain embodiments, trichothecene mycotoxins comprises deoxynivalenol (DON), 15-acetyl-deoxynivalenol (15-ADON), 3-acetyl-deoxynivalenol (3-ADON), nivalenol (NIV), fusarenon-X (Fus-X), diacetoxyscirpenol (DAS), T-2 toxin (T-2), and HT-2 toxin (HT-2).

Herein, the term “epoxy group-removing catalytic activity” refers to an activity or function of removing an epoxy group (preferably the epoxy group formed between the 12-position carbon and the 13-position carbon) in a trichothecene mycotoxin. The specific catalytic process is as follows:

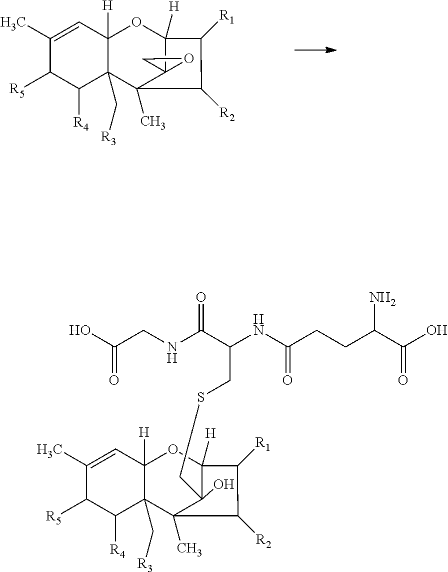

wherein R₁ to R₅ have the same meanings as in the general formulae (I) and (II).

### EXAMPLES

I. Preparation of FTCD Active Polypeptide

1. Materials and Methods

Escherichia coli DH5a strain, expression strain BL21 (DE3), prokaryotic expression vector pET-28a(+) and plasmid pMD19-T-FTCD were preserved in our laboratory, wherein plasmid pMD19-T-FTCD contained a de-epoxidase gene derived from Thinopyrum, the sequence of which was shown in SEQ ID NO: 36.

1.2 Experimental Methods

1.2.1 The Recombinant Expression Vector pET28a-FTCD was Constructed by the Following Method.

The primers with NcoI and BamHI restriction sites were designed according to the sequence of expression vector pET28a, and the primer sequences were as follows (underlined sequences indicate the restriction sites):

Forward primer:
5′-CCATGGCTAGAAATCCACCCATCGTCATCACC-3′
Reverse primer:
5′-GGATCCTCTTCACCTCGGCATACTTGTC-3′

PCR amplification was performed using plasmid pMD19-T-FTCD as a template. The amplification product was detected by 1% agarose gel electrophoresis, and a target fragment was recovered by cutting the gel; the target fragment and pET28a vector were digested by double enzymes, NcoI and BamHI, respectively, followed by gel recovery and ligation with T4 ligase; the ligation product was transformed into Escherichia coli DH5a, and colony PCR and double digestion identification were performed to obtain a target gene of about 900 bp and pET28a vector backbone of about 5,000 bp. Further sequencing was performed to verify that the sequence and the reading frame of the recombinant expression vector pET28a-FTCD were correct.

1.2.2 Induced Expression of Polypeptides

The recombinant expression vector plasmid pET28a-FTCD was transformed into the competent cells of Escherichia coli expression strain BL21(DE3); after PCR detection, the positive monoclones on transformation plates were picked and inoculated into test tubes containing 50 μg/mL Kana in 3 mL of LB liquid medium, and shaken at 37° C. at 220 r/min overnight. The next day, the culture was inoculated into a Kana LB liquid medium and shaken until the OD600 of the bacterial cells was 0.6 to 0.8. 1 mL of the culture was taken out and centrifuged at room temperature for 2 min, the supernatant was discarded, and the bacterial pellet was resuspended in 100 μl of 1× loading buffer. IPTG was added to the remaining culture to a final concentration of 0.5 mM, and the fusion protein was induced to express by shaking at 37° C. at 220 r/min for 4 h. 1 mL of the culture was taken out and centrifuged at 10,000 r/min for 2 min at room temperature, the supernatant was discarded, and the bacterial pellet was resuspended in 100 μl of 1× loading buffer. The remaining culture was centrifuged at 4,000 r/min for 10 min, the supernatant was discarded, and the bacterial pellet was resuspended in PBS; after the resuspension solution was treated by ultrasonication, the supernatant and the pellet were taken and added to the loading buffer to resuspend respectively.

1.2.3 Purification of Polypeptides

The protein solution was purified using Ni column and collected using a low pressure chromatography system, and added to a dialysis bag for overnight dialysis against 50 mM Tris-HCl, 0.30 M NaCl, pH 8.0.

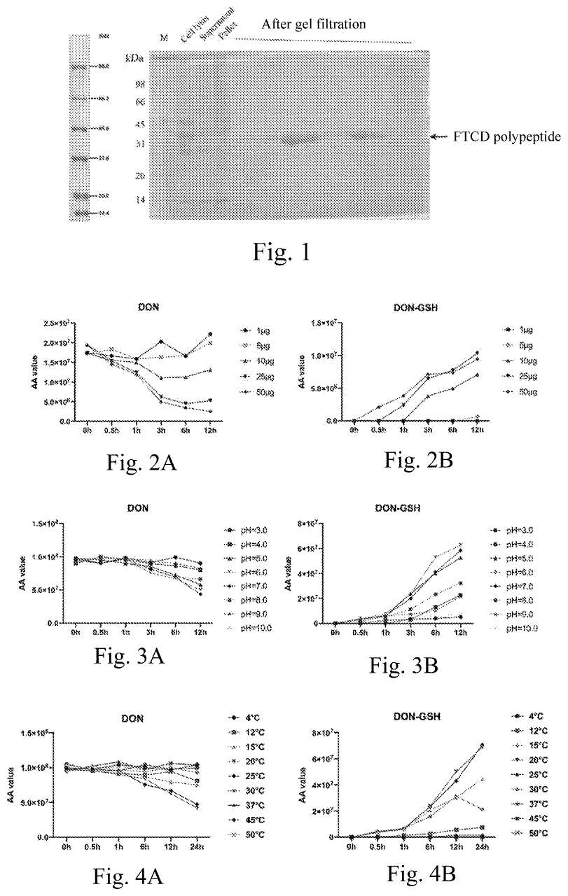

The dialyzed product was shaken at 37° C. for 4 h to induce protein expression with 0.5 mmol/L IPTG, and the bacterial cells were collected and resuspended in PBS. After ultrasonication, the supernatant was collected, and the supernatant was purified by a Ni column and a molecular sieve. The results of SDS-PAGE electrophoresis showed that a polypeptide in the form of soluble protein was obtained, with a molecular weight of about 33 kDa, and the purified protein had a single band, indicating that the purification effect was good (see FIG. 1).

II. Establishment of an In Vitro Enzymatic Reaction System of Polypeptide

1. Experimental Methods:

1.1 Reagent: 0.5 mg/ml Trichothecenes (DON, 3DON, 15ADON, FUS-X, NIV, T2, H-T2, and DAS) Prepared by Adding Distilled Water to 1 mg of Trichothecenes to 2 ml, Filtered and Sterilized.

1.2 Establishment of an In Vitro Enzymatic Reaction System

The optimal conditions for the in vitro enzymatic reaction system of FTCD polypeptide were established by gradient experiments of three different factors affecting the enzymatic reaction:

(1) the gradient of reaction enzyme amounts: 1 μg, 5 μg, 10 μg, 25 μg, and 50 μg;

(2) the pH gradient set with various buffers: ranging from 3.0 to 10.0, disodium hydrogen phosphate-citric acid buffer (pH=3.0, 4.0, 5.0), disodium hydrogen phosphate-potassium dihydrogen phosphate buffer (pH=6.0, 7.0), and Tris-phosphate buffer (pH=8.0, 9.0, 10.0); and

(3) the gradient of reaction temperatures: 4° C., 12° C., 15° C., 20° C., 25° C., 30° C., 37° C., 45° C., and 50° C.

2. Experimental Results:

2.1 Effect of Enzyme Amount on the Enzymatic Reaction System

The reaction was performed in a phosphate buffer (PBS) (pH=7.0), at 25° C. for 12 h, and samples were taken at 0 h, 0.5 h, 1 h, 3 h, and 6 h respectively for LC-HRMS analysis; through the area results of first-level scanning of LC-HRMS, the changes in the contents of the two substances, DON as the reaction substrate and the GSH adduct as the reaction product, were obtained with proceeding of reaction, so as to obtain the optimal enzyme amount for the reaction, as shown in FIGS. 2A and 2B.

The experimental results obtained by changing the enzyme amount showed that when the enzyme amount was 1 to 25 μg, the amount of DON-GSH produced was positively correlated with the amount of enzyme added within the same time period. When the enzyme amount exceeded 25 μg, the amount of DON-GSH produced tended to be stable. Therefore, 25 μg was chosen as the optimal test enzyme amount.

2.2 Effect of pH of the Reaction System on the Enzymatic Reaction System

The experimental results of the pH gradient of the enzymatic reaction buffer were shown in FIGS. 3A and 3B. FIGS. 3A and 3B show that when the pH of the buffer was 6.0, the amount of the product DON-GSH reached the highest value, while the content of the reaction substrate DON was the lowest, and thus the suitable pH of the buffer was between 5.0 and 7.0.

3. Effect of Reaction Temperature on the Enzymatic Reaction System

According to the above experimental results, under the conditions at the pH of the reaction buffer of 7.0 and the addition amount of enzyme of 25 μg, the temperatures were set at 4° C., 12° C., 15° C., 20° C., 25° C., 30° C., 37° C., 45° C., and 50° C., and the reaction time was 24 h; samples were taken at 0 h, 0.5 h, 1 h, 6 h, 12 h, and 24 h respectively for LC-HRMS analysis; through the area results of first-level scanning of LC-HRMS, the changes in the contents of the two substances, DON as the reaction substrate and the GSH adduct as the reaction product, were obtained with proceeding of reaction, so as to obtain the optimal temperature for the reaction.

The results of experiments obtained by setting different reaction temperatures were shown in FIGS. A and 4B. FIGS. 4A and 4B show that the difference in the effect on the enzymatic reaction was not significant at 20° C. to 25° C., and the content of the product can all reach the maximum value; the amount of DON-GSH produced decreased with decreasing temperature below 15° C.; the amount of DON-GSH produced was inversely correlated with the increase of reaction temperature at 30° C. to 37° C.; the product DON-GSH can not be detected by first-level scanning of LC-HRMS above 37° C., indicating that the enzyme had basically lost its activity. Therefore, the condition at 20° C. to 25° C. was more suitable for the enzymatic reaction.

The above experimental results showed that the most suitable conditions to carry out in vitro enzymatic reaction were as follows: in the reaction system, 25 μg of purified FTCD protein was added, and after adding an appropriate amount of reaction substrates, the system was supplemented to 200 μl with a buffer at a pH of 5.0 to 7.0, mixed, and reacted at 20° C. to 25° C.

### III. The Reaction of Removing Epoxy Groups of Trichothecene Mycotoxins Catalyzed by Active Polypeptide FTCD

1. Experimental Methods:

1.1 In Vitro Enzymatic Reaction:

DON, 3-DON, 15-ADON, NIV, DAS, HT-2, and T-2 toxins (1 mg) were dissolved in freshly prepared GSH (30.7 mg, 100 μmol in PBS buffer respectively, and the enzyme was added, and incubated in a water bath at 20° C. for 24 h.

1.2 LC-HRMS (/MS) analysis

The in vitro reaction solution was filtered through a 0.22 μm filter membrane, and transferred to an injection vial for LC-HRMS detection.

Thermo Scientific™ Q Exactive™ Hybrid Quadrupole Orbitrap Mass Spectrometer was used. A UHPLC system (Accela, Thermo Fisher Scientific, San Jose, Calif., USA) was used in conjunction with an Orbitrap equipped with an electrospray ionization (ESI) source. Chromatography was performed on a reverse phase XBridge C18, with an inner diameter of 150><2.1 mm, and a particle size of 3.5 μm (Waters, Dublin, Ireland), at a column temperature of 35° C. The flow rate was 300 μL min⁻¹, and the injection volume was 3 μL. U3000 liquid chromatograph was used with the following conditions: mobile phase: A: 0.1% aqueous acetic acid, B: acetonitrile; elution gradient: A=90% at 0 to 0.2 min; A gradually decreased to 10% at 0.2 to 6 min; A=10% at 6 to 8 min; A gradually increased to 90% at 8.1 min; and A=90% at 8.1 to 10 min.

(1) Full scan mode: This mode rapidly performed alternated positive and negative ion scans in the m/z range of 200 to 1000. The ESI interface in positive ion mode was set as follows: sheath gas: 40; auxiliary gas: 10; capillary voltage: 3.8 kV; and capillary temperature: 350° C. The AGC target was set to 2><e5. The ESI interface in negative ion mode was set to 2.9 kV; sheath gas: 4; and auxiliary gas: 0. The resolution in this mode was set to 70,000.

(2) The liquid chromatography method and chromatographic conditions in Full scan+ddms (first-level full scan+automatic triggering of second-level) mode were the same as above. In this method, full scan and MS2 scan were used alternately with normalized collision energy set to 20 eV and resolution set to 17,500 during product ion scanning

(3) PRM mode can be used to quantify the relative abundance of toxins and their derivatives in a sample. After screening of precursor ions in PRM mode, dissociation was induced at normalized collision energy (HCID), followed by fragment detection of product ions in Orbitrap with a resolution set to 17,500. Normalized collision energies were used, with collision energies applied (15, 30 and 45 eV) being dependent on the specific analyte.

Xcalibur 2.1.0 (Thermo Fisher Scientific, San Jose, Calif., USA) were used for analysis of data of LC-HRMS (/MS). Extracted ion chromatograms (EICs) of toxins and their derivatives were investigated using the extracted chromatographic peak shape, retention time (±0.2 min) and mass (±5 ppm) of the bioconversion products. According to secondary spectra and basic structures of the substances, the neutral loss was analyzed, and chemical structures were inferred.

2. Experimental Results

2.1 Catalyzing and Converting DON Toxin to Glutathione Adduct DON-GSH by FTCD

FIG. 5A shows extracted ion chromatograms (EICs) of in vitro enzymatic reaction of DON and GSH by LC-HRMS₁ (Method 1). As shown in FIG. 5A, the extracted ion chromatograms (EICs) of DON were obtained by LC-HRMS (Full scan mode) in negative ion mode, with an m/z of 355.13984 (corresponding to [M+CH₃COO]⁻ form, Δ±5 ppm); the DON-GSH adduct was detected in positive ion mode, with an m/z of 604.21707 (corresponding to [M+H]⁺, Δ±5 ppm).

FIG. 5B shows an LC-HRMS₂ (Method 2) mass spectrogram of the product ions produced by the high-energy collision induced dissociation of DON-GSH obtained by in vitro enzymatic reaction of DON and GSH, in [M+H]⁺ (m/z 604.21707, Δ±5 ppm). The MS fragment of the DON-GSH epoxy adduct was investigated by targeted HRMS₂ analysis of positively charged ([M+H]⁺) ions. Ion fragmentation of DON-GSH yielded a characteristic ion with an m/z of 299.0939, corresponding to C₁₄H₁₉O₅S⁺. This characteristic ion can be attributed to cleavage of the side chain at C-6 and loss of GSH moiety other than S. This fragment can also be further cleaved to yield ions with m/z ratios of 281.08482 (C₁₄H₁₇O₄S⁺), 263.07425 (C₁₄H₁₅O₃S⁺) and 231.10218 (C₁₄H₁₅O₃⁺). The product ion with an m/z of 263.07425 was the base peak of the HRMS₂ mass spectrogram, and this product ion was generated by removing of two molecules of H₂O based on the ion with an m/z of 299.0939.

After the loss of glycine in DON-GSH, a fragment ion with an m/z of 529.18503 (C₂₃H₃₃O₁₀N₂S⁺) can be obtained, and a fragment ion with an m/z of 475.17466 (C₂₀H₃₁O₉N₂S⁺) can also be obtained after the loss of anhydroglutamic acid. The ion fragment with the side chain at C-6 lost, with an m/z of 574.20717 (C₂₄H₃₆O₁₁N₃S⁺), can generate a characteristic ion (C₁₉H₂₉O₈N₂S⁺) with an m/z of 445.16389 after the loss of anhydroglutamic acid from the GSH moiety; and can also generate an ion with an m/z of 428.13733 (C₁₉H₂₆O₈NS⁺) after removing of glutamine.

The product ion had an m/z of 308.09108 (C₁₀H₁₈O₆N₃S⁺, corresponding to [M+H]⁺ of GSH). This fragment ion lost anhydroglutamic acid to obtain an ion with an m/z of 179.04907 (C₅H₁₁O₃N₂S⁺); and lost glutamine to obtain an ion with an m/z of 162.02251 (C₅H₉O₃NS⁺). In addition, the product ions with m/z ratios of 130.05044 (C₅H₈O₃N⁺) and 145.06077 (C₅H₉O₃N₂⁺) were associated with GSH.

2.2 Catalyzing and Converting 3-ADON Toxin to Glutathione Adduct 3-ADON-GSH by FTCD

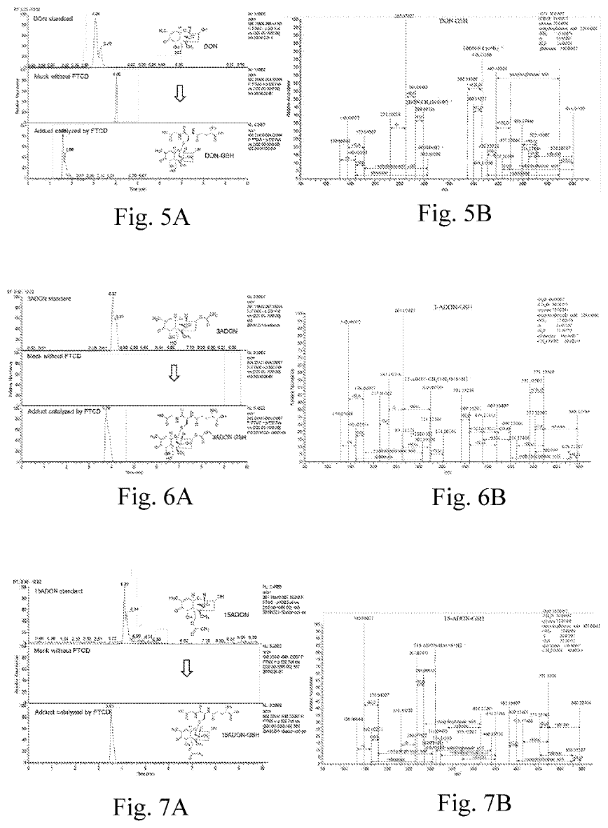

FIG. 6A shows extracted ion chromatograms (EICs) of in vitro enzymatic reaction of 3-ADON and GSH by LC-HRMS (Method 1). As shown in FIG. 6A, the extracted ion chromatograms (EICs) of 3-ADON were obtained by LC-HRMS (Full scan mode) in negative ion mode, with an m/z of 397.15041 (corresponding to [M+CH₃COO]⁻ form, Δ±5 ppm); the 3-ADON-GSH adduct was detected in positive ion mode, with an m/z of 646.22764 (corresponding to [M+H]⁺, Δ±5 ppm).

FIG. 6B shows an LC-HRMS₂ (Method 2) mass spectrogram of the product ions produced by the high-energy collision induced dissociation of 3-ADON-GSH obtained by in vitro enzymatic reaction of 3-ADON and GSH, in [M+H]+ (m/z 646.22764, Δ±5 ppm). Targeted HRMS₂ analysis was performed on the positively charged ([M+H]⁺) 3-ADON-GSH epoxy adduct ion: ion fragmentation of 3-ADON-GSH yielded a characteristic ion with an m/z of 323.09539, corresponding to C₁₆H₁₉O₅S⁺. This characteristic ion can be attributed to cleavage of the side chain attached at C-6, dehydration, and loss of GSH moiety other than S. This fragment can also be further cleaved to yield ions with m/z ratios of 263.07425 (C₁₄H₁₅O₃S⁺) and 231.10218 (C₁₄H₁₅O₃⁺). The product ion with an m/z of 263.07425 was the base peak of the HRMS₂ mass spectrogram, and this product ion was generated by removing of CH3COOH at C-3 based on the ion with an m/z of 323.09539.

After the loss of glycine in 3-ADON-GSH, a fragment ion with an m/z of 571.19560 (C₂₅H₃₅O₁₁N₂S⁺) can be obtained, and by further fragmentation of the side chain at C-6, a fragment ion with an m/z of 541.18503 (C₂₄H₃₃O₁₀N₂S⁺) can be yielded. A fragment ion m/z 628.21707 (C₂₇H₃₈O₁₂N₃S⁺) obtained by removing of 1 molecule of H₂O can generate an ion with an m/z of 553.18503 (C₂₅H₃₃O₁₀N₂S⁺) after the loss of glycine, and also generate an ion with an m/z of 499.17466 (C₂₂H₃₁O₉N₂S⁺) after the loss of anhydroglutamic acid.

2.3 Catalyzing and Converting 15-ADON Toxin to Glutathione Adduct 15-ADON-GSH by FTCD

FIG. 7A shows extracted ion chromatograms (EICs) of in vitro enzymatic reaction of 15-ADON and GSH by LC-HRMS (Method 1). As shown in FIG. 7A, the extracted ion chromatograms (EICs) of 15-ADON were obtained by LC-HRMS (Full scan mode) in negative ion mode, with an m/z of 397.15041 (corresponding to [M+CH₃COO]⁻ form, Δ±5 ppm); the 15-ADON-GSH adduct was detected in positive ion mode, with an m/z of 646.22764 (corresponding to [M+H]⁺, Δ±5 ppm).

FIG. 7B shows an LC-HRMS₂ (Method 2) mass spectrogram of the product ions produced by the high-energy collision induced dissociation of 15-ADON-GSH obtained by in vitro enzymatic reaction of 15-ADON and GSH, in [M+H]+ (m/z 646.22764, Δ±5 ppm). The MS fragment of the 15-ADON-GSH epoxy adduct was investigated by targeted HRMS₂ analysis of positively charged ([M+H]⁺) ions. Ion fragmentation of 15-ADON-GSH yielded a characteristic ion with an m/z of 311.09475, corresponding to C₁₅H₁₉O₅S⁺. This characteristic ion can be attributed to cleavage of the side chain CH₃COOH attached at C-15 and loss of GSH moiety other than S.

Like the case of 3-ADON-GSH, after the loss of glycine in 15-ADON-GSH, a product ion with an m/z of 571.1956 (C₂₅H₃₅O₁₁N₂S⁺) can be obtained. An ion with an m/z of 628.21707 (C₂₇H₃₈O₁₂N₃S⁺) obtained by removing of 1 molecule of H₂O can generate an ion with an m/z of 553.18503 (C₂₅H₃₃O₁₀N₂S⁺) after the loss of glycine. An ion with an m/z of 499.17466 (C₂₂H₃₁O₉N₂S⁺) can be obtained after the loss of anhydroglutamic acid.

The characteristic ion with an m/z of 440.13736 (C₂₀H₂₆O₈NS⁺) can generate a fragment ion with an m/z of 311.09475 (C₁₅H₁₉O₅S⁺) after the loss of anhydroglutamic acid. The characteristic ion with an m/z of 450.15471 (C₁₇H₂₈O₉N₃S⁺) generated a product ion with an m/z of 375.12267 (C₁₅H₂₃O₇N₂S⁺) after the loss of glycine; also generated an ion with an m/z of 321.1121 (C₁₂H₂₁O₆N₂S⁺) after the loss of anhydroglutamic acid; and in addition, the characteristic ion can further generate a product ion with an m/z of 414.13295 (C₁₇H₂₄O₇N₃S⁺) after removing two molecules of H₂O, and this product ion can further generate an ion with an m/z of 339.10091 (C₁₅H₁₉O₅N₂S⁺) after the loss of glycine, and also generate an ion with an m/z of 285.09035 (C₁₂H₁₇O₄N₂S⁺) after the loss of anhydroglutamic acid that can further generate an ion with an m/z of 267.07979 (C₁₂H₁₅O₃N₂S⁺) after dehydration. The characteristic ion with an m/z of 145.06077 (C₅H₉O₃N₂⁺, Δ±5 ppm) associated with GSH was the base peak of the mass spectrogram.

2.4 Catalyzing and Converting NIV Toxin to Glutathione Adduct NIV-GSH by FTCD

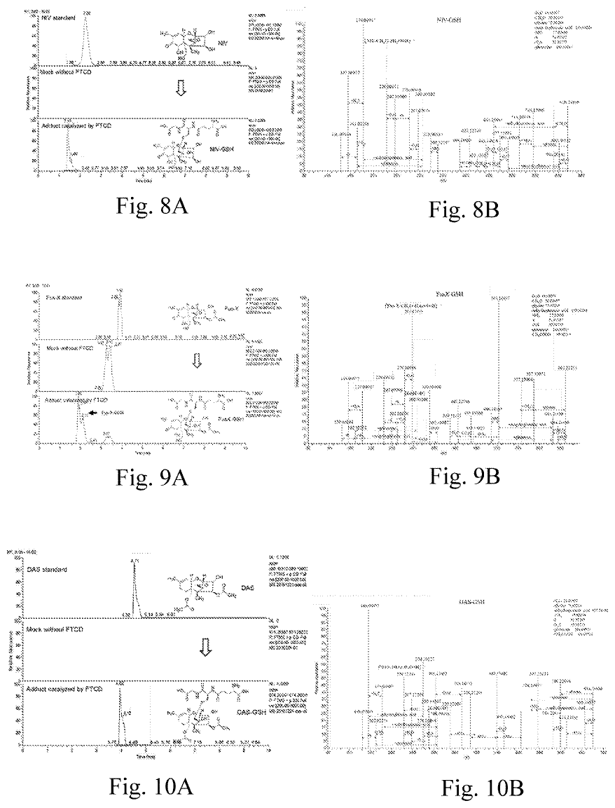

As shown in FIG. 8A, the extracted ion chromatograms (EICs) of NIV were obtained by LC-HRMS (Full scan mode) in negative ion mode, with an m/z of 371.13366 (corresponding to [M+CH₃COO]⁻ form, Δ±5 ppm); the NIV-GSH adduct was detected in positive ion mode, with an m/z of 620.21199 (corresponding to [M+H]⁺, Δ±5 ppm).

FIG. 8B shows an LC-HRMS₂ (Method 2) mass spectrogram of the product ions produced by the high-energy collision induced dissociation of NIV-GSH obtained by in vitro enzymatic reaction of NIV and GSH, in [M+H]⁺ (m/z 620.21199, Δ±5 ppm). The MS fragment was investigated by targeted HRMS₂ analysis of positively charged ([M+H]⁺) NIV-GSH epoxy adduct ions. Ion fragmentation of NIV-GSH yielded a product ion with an m/z of 229.08652, corresponding to C₁₄H₁₃O₃⁺. This product ion can be attributed to cleavage of the side chain at C-6, break of 3 molecules of H₂O and loss of GSH moiety, and this structure retained the basic backbone of NIV.

After the loss of glycine in NIV-GSH, a product ion with an m/z of 545.17995 (C₂₃H₃₃O₁₁N₂S⁺) can be obtained. A product ion with an m/z of 491.16938 (C₂₀H₃₁O₁₀N₂S⁺) can also be obtained after the loss of anhydroglutamic acid. An ion with an m/z of 590.20142 (C₂₄H₃₆O₁₂N₃S⁺) was obtained after the cleavage of the side chain at C-6; and after the GSH moiety of this ion lost anhydroglutamic acid, a product ion with an m/z of 461.15881 (C₁₉H₂₉O₉N₂S⁺) can be obtained.

The GSH in the form of [M+H]⁺ can generate a product ion with an m/z of 162.02251 (C₅H₉O₃NS⁺) after the loss of glutamine; and can also generate an ion with an m/z of 179.04907 (C₅H₁₁O₃N₂S⁺) after the loss of anhydroglutamic acid, which was the most prominent product ion in the HRMS₂ mass spectrogram. In addition, both the product ion with an m/z of 130.05044 (C₅H₈O₃N⁺) and the product ion with an m/z of 145.06077 (C₅H₉O₃N₂⁺) were associated with GSH.

2.5 Catalyzing and Converting Fus-X Toxin to Glutathione Adduct Fus-X-GSH by FTCD

FIG. 9A shows extracted ion chromatograms (EICs) of in vitro enzymatic reaction of Fus-X and GSH by LC-HRMS (Method 1). As shown in FIG. 9A, the extracted ion chromatograms (EICs) of Fus-X were obtained by LC-HRMS (Full scan mode) in negative ion mode, with an m/z of 377.12069 (corresponding to [M+Na]⁺ form, Δ±5 ppm); the Fus-X-GSH adduct was detected in positive ion mode, with an m/z of 662.22255 (corresponding to [M+H]+, Δ±5 ppm).

FIG. 9B shows an LC-HRMS₂ (Method 2) mass spectrogram of the product ions produced by the high-energy collision induced dissociation of Fus-X-GSH obtained by in vitro enzymatic reaction of Fus-X and GSH. The MS fragment was investigated by targeted HRMS₂ analysis of positively charged ([M+H]⁺) FusX-GSH epoxy adduct ions. Ion fragmentation of FusX-GSH yielded a product ion with an m/z of 297.07973, corresponding to C₁₄H₁₇O₅S⁺. This product ion can be attributed to cleavage of the side chain at C-4, cleavage of the side chain at C-6 and loss of GSH moiety other than S, and this structure retained only the basic backbone of Fus-X.

After the loss of glycine in FusX-GSH, a product ion with an m/z of 587.19051 (C₂₅H₃₅O₁₂N₂S⁺) can be obtained. A characteristic ion with an m/z of 632.21198 (C₂₆H₃₈O₁₃N₃S⁺) formed after cleavage of the side chain at C-6 can generate a product ion with an m/z of 503.16937 (C₂₁H₃₁O₁₀N₂S⁺) after the loss of anhydroglutamic acid, and also generate an ion with an m/z of 486.14281 (C₂₁H₂₈O₁₀NS⁺) after the loss of glutamine. The product ion with an m/z of 503.16937 (C₂₄H₃₆O₁₂N₃S⁺) was the most prominent product ion in the HRMS₂ mass spectrogram.

The GSH in the form of [M+H]⁺ can generate a product ion with an m/z of 162.02251 (C₅H₉O₃NS⁺) after the loss of glutamine; and can also generate an ion with an m/z of 179.04907 (C₅H₁₁O₃N²S⁺) after the loss of anhydroglutamic acid. In addition, both the product ion with an m/z of 130.05044 (C₅H₈O₃N⁺) and the product ion with an m/z of 145.06077 (C₅H₉O₃N₂⁺) were associated with GSH.

2.6 Catalyzing and Converting DAS Toxin to Glutathione Adduct DAS-GSH by FTCD

FIG. 10A shows extracted ion chromatograms (EICs) of in vitro enzymatic reaction of DAS and GSH by LC-HRMS (Method 1). The extracted ion chromatograms (EICs) of DAS was obtained by LC-HRMS (Full scan mode) in positive ion mode, with an m/z of 389.15707 (corresponding to [M+Na]+form, Δ±5 ppm); and DAS-GSH adduct was detected with an m/z of 674.25894 (corresponding to [M+H]⁺, Δ±5 ppm).

FIG. 10B shows an LC-HRMS₂ (Method 2) mass spectrogram of the product ions produced by the high-energy collision induced dissociation of DAS-GSH obtained by in vitro enzymatic reaction of DAS and GSH, in [M+H]⁺ (m/z 674.25894, Δ±5 ppm). The MS fragment of the DAS-GSH epoxy adduct was investigated by targeted HRMS₂ analysis of positively charged ([M+H]⁺) ions. Ion fragmentation of DAS-GSH yielded a product ion with an m/z of 229.12231, corresponding to C₁₅H₁₇O₂⁺. This product ion can be attributed to cleavage of the side chain CH3COOH attached at C-4 and C-15, dehydration, and loss of GSH moiety.

DAS-GSH can generate a product ion with an m/z of 599.22690 (C₂₇H₃₉O₁₁N₂S⁺) after the loss of glycine; a product ion with an m/z of 528.18977 (C₂₄H₃₄O₁₀NS⁺) after the loss of glutamine; a product ion with an m/z of 545.21633 (C₂₄H₃₇O₁₀N₂S⁺) after the loss of anhydroglutamic acid; and also a characteristic ion with an m/z of 614.23781 (C₂₇H₄₀O₁₁N₃S⁺) after the loss of CH3COOH.

Among the product ions with m/z ratios of 130.05044 (C₅H₈O₃N⁺), 145.06077 (C₅H₉O₃N₂⁺), 162.02251 (C₅H₉O₃NS⁺), and 179.04907 (C₅H₁₁O₃N₂S⁺) associated with GSH, the characteristic ion with an m/z of 179.04907 (C₅H₁₁O₃N₂S⁺) obtained after the loss of anhydroglutamic acid was the base peak of the mass spectrogram.

2.7 Catalyzing and Converting HT-2 to Glutathione Adduct HT-2-GSH by FTCD

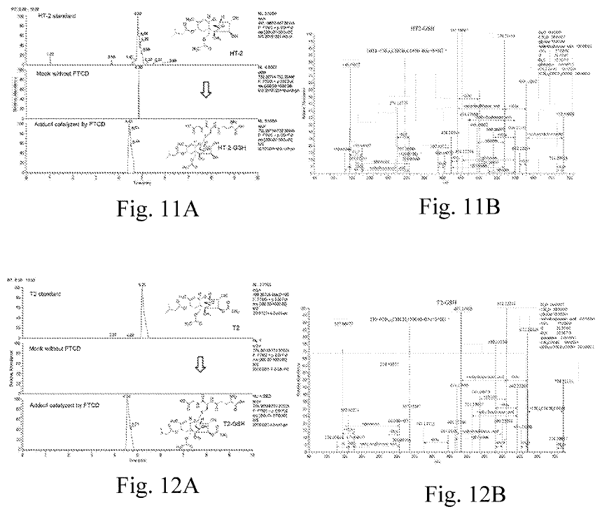

FIG. 11A shows extracted ion chromatograms (EICs) of in vitro enzymatic reaction of HT-2 and GSH by LC-HRMS (Method 1). The extracted ion chromatograms (EICs) of HT-2 was obtained by LC-HRMS (Full scan mode) in positive ion mode, with an m/z of 447.19894 (corresponding to [M+Na]+form, Δ±5 ppm); and HT-GSH adduct was detected with an m/z of 732.30080 (corresponding to [M+H]⁺, Δ±5 ppm).

FIG. 11B shows an LC-HRMS₂ (Method 2) mass spectrogram of the product ions produced by the high-energy collision induced dissociation of HT-GSH adduct obtained by in vitro enzymatic reaction of HT-2 and GSH, in [M+H]+ (m/z 732.30080, Δ±5 ppm). The MS fragment was investigated by targeted HRMS₂ analysis of positively charged ([M+H]⁺) HT-GSH epoxy adduct ions. Fragmentation of HT-GSH yielded a product ion with an m/z of 295.10048, corresponding to C₁₅H₁₉O₄S⁺. This product ion can be attributed to cleavage of (CH₃)₂CHCH₂COOH at C-8, cleavage of CH3COOH at C-15 and loss of GSH moiety other than S, and this structure retained the basic backbone of HT-2. Furthermore, the ion with an m/z of 274.10335 was generated due to the neutral loss of H₂S resulting from cleavage of the —SH bond of GSH in the form of [M+H]⁺.

A characteristic ion with an m/z of 570.21226 (C₂₅H₃₆O₁₀N₃S⁺) can be obtained after the cleavage of side chains of HT-GSH at C-8 and C-15, and this ion can generate a fragment ion with an m/z of 495.18022 (C₂₃H₃₁O₈N₂S⁺) after the loss of glycine, a fragment ion with an m/z of 441.16965 (C₂₀H₂₉O₇N₂S⁺) after the loss of anhydroglutamic acid, and also a fragment ion with an m/z of 424.14309 (C₂₀H₂₆O₇NS⁺) after the loss of glutamine. The base peak of the mass spectrogram was at m/z of 441.16965.

In addition, ions with m/z ratios of 130.05044 (C₅H₈O₃N⁺), 145.06077 (C₅H₉O₃N₂⁺), 162.02251 (C₅H₉O₃NS⁺), and 179.04907 (C₅H₁₁O₃N₂S⁺) associated with GSH were also detected.

2.8 Catalyzing and Converting T-2 to Glutathione Adduct T-2-GSH by FTCD

FIG. 12A shows extracted ion chromatograms (EICs) of in vitro enzymatic reaction of T-2 and GSH by LC-HRMS (Method 1). The extracted ion chromatograms (EICs) of T-2 was obtained by LC-HRMS (Full scan mode) in positive ion mode, with an m/z of 489.20950 (corresponding to [M+Na]⁺ form, Δ±5 ppm); and T2-GSH adduct was detected with an m/z of 774.31136 (corresponding to [M+H]⁺, Δ±5 ppm).

FIG. 12B shows an LC-HRMS₂ (Method 2) mass spectrogram of the product ions produced by the high-energy collision induced dissociation of T2-GSH adduct obtained by in vitro enzymatic reaction of T-2 and GSH, in [M+H]+ (m/z 774.31136, Δ±5 ppm). The MS fragment was investigated by targeted HRMS₂ analysis of positively charged ([M+H]⁺) T2-GSH epoxy adduct ions. Fragmentation of T2-GSH yielded a product ion with an m/z of 337.11105, corresponding to C₁₇H₂₁O₅S⁺. This product ion can be attributed to cleavage of the side chains attached at C-8 and C-15 and loss of GSH moiety other than S, and this structure retained the basic backbone of T-2. Furthermore, the ion with an m/z of 274.10335 was generated due to the neutral loss of H₂S resulting from cleavage of the —SH bond of GSH in the form of [M+H]⁺.

The characteristic ion with an m/z of 612.22283 (C₂₇H₃₈O₁₁N₃S⁺) obtained after the cleavage of the side chains of T2-GSH at C-8 and C-15 was the base peak of the mass spectrogram. This ion can generate a fragment ion with an m/z of 537.19079 (C₂₅H₃₃O₉N₂S⁺) after the loss of glycine; a fragment ion with an m/z of 483.18022 (C₂₂H₃₁O₈N₂S⁺) after the loss of anhydroglutamic acid; and also a fragment ion with an m/z of 466.15366 (C₂₂H₂₈O₈NS⁺) after the loss of glutamine.

Like the case of HT2-GSH, ions with m/z ratios of 130.05044 (C₅H₈O₃N⁺), 145.06077 (C₅H₉O₃N₂⁺), 162.02251 (C₅H₉O₃NS⁺), and 179.04907 (C₅H₁₁O₃N₂S⁺) associated with GSH were detected.

3. Experimental Conclusion

The active polypeptide of the present invention can efficiently catalyze trichothecene mycotoxins (comprising DON, 3DON, 15ADON, FUS-X, NIV, T2, H-T2, DAS, and the like) into glutathione adducts in vitro, and it can be seen from the secondary spectrum that the formation of the adducts destroyed the epoxy ring structure playing a major role in the toxicity of trichothecenes, which can greatly reduce the toxicity of the toxins.

IV. Cytotoxicity Test of Trichothecene Mycotoxin-GSH Derivatives

1. Cell Culture

Using a DMEM basal medium supplemented with 10% fetal bovine serum and 500 μl of penicillin-streptomycin (double antibiotics), the pancreatic cancer cell line (PATU8988), human embryonic kidney cell 293-derived line (293T) and normal human esophageal epithelial cells (HEECs) were cultured in a thermostatic incubator with 5% CO2 at 37° C. When the cells grew to 80% to 90% adherent to the wall of the flask, they were subcultured every 2 to 3 d, and the cells were collected by trypsinization and subcultured. According to the cell growth state, cells at the logarithmic growth stage were selected for experiments.

2. Cytotoxicity Assay by CCK8 Method

The Cell Counting Kit-8 (CCK-8 for short) reagent can be used to analyze cell proliferation and cytotoxicity. The three cell lines at the logarithmic growth stage were inoculated into 96-well plates with 100 ul (about 5×10³ cells) per well, and were routinely cultured for 24 h at 37° C. with 5% CO2. The medium was discarded and grouped. Wells were set in triplicate for each group for observation, and the treatment methods of each group were as follows: the blank group was the zero-adjustment well containing medium only, the control group was the DMEM medium containing 10% fetal bovine serum, and gradients of low, medium and high concentrations were all set for trichothecenes and their corresponding glutathione adducts produced by the enzymatic reaction. After culturing at 37° C. for 48 h, 10 ul of CCK8 solution was added to each well to continue the culture. After 2 h, the culture supernatants in the wells were carefully pipetted and discarded, the OD value of each well was measured by a full-wavelength multi-functional microplate reader at a wavelength of 450 nm, and the cell viability was calculated.

3. Experimental Results

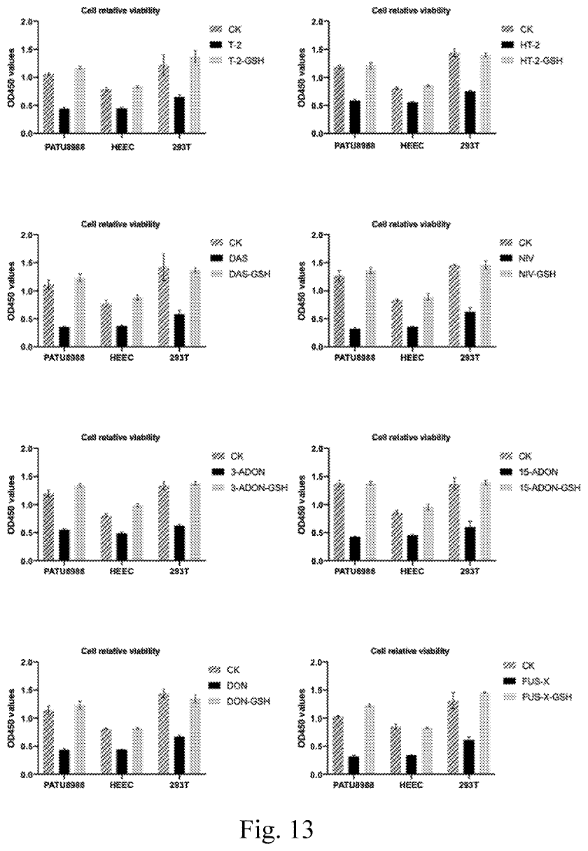

The cells were plated at a concentration of 5×10⁷ L⁻¹, and the OD450 values for the pancreatic cancer cell line (PATU8988), human embryonic kidney cell 293-derived line (293T) and normal human esophageal epithelial cells (HEEC) were detected using a CCK-8 microplate reader after 48 h treatment with trichothecenes and their corresponding glutathione adducts produced by the enzymatic reaction. Wells were set in triplicate for each group for observation, and the treatment methods of each group were as follows: the blank group was the zero-adjustment well containing medium only, the control group was the DMEM medium containing 10% fetal bovine serum, and trichothecenes and their corresponding glutathione adducts produced by the enzymatic reaction were provided at corresponding concentrations according to the results in literatures for treatment. The results were shown in FIG. 13.

As shown in FIG. 13, the viability of PATU8988, 293T and HEEC decreased sharply after treatment with corresponding concentrations of trichothecenes (DON, 3-ADON, 15-ADON, FUS-X, NIV, T-2, HT-2, and DAS) for 48 h, indicating that different trichothecenes are highly toxic to cells; while the treatment with corresponding derivatives of trichothecenes (DON, 3-ADON, 15-ADON, FUS-X, NIV, T-2, HT-2, and DAS) produced by the reaction led to substantially the same cell viability as the blank control at the corresponding same concentrations, indicating that the glutathione adducts corresponding to the above 8 trichothecenes had substantially no toxic effect on cells.

Through the above experiments, it was found that all the trichothecenes can have a strong inhibitory effect on the cell viability, while the corresponding glutathione adducts of most trichothecenes had almost no effect on cell viability at the same mass concentrations. In conclusion, the production of glutathione adducts of trichothecenes can greatly reduce the toxic effects of these trichothecenes on cells.

V. Research on Host Cells Expressing the FTCD Active Polypeptide and its Function

1. Construction of yeast expression plasmid pPICZαA-FTCD

The cDNA of the de-epoxidase gene derived from Thinopyrum ponticum had a length of 865 bp (SEQ ID NO: 36), the sequence did not comprise Bsp119I and XbaI restriction sites, and the primer sequences were designed as follows:

F:
5′-ATTATTCGAAAGAAATCCACCCATCGTCATCACC-3′
R:
5′-TTGTTCTAGACTACTTCACCTCGGCATACTTGTC-3′

The underlined portions are restriction endonuclease sites. The whole gene sequence of the cDNA was obtained by PCR. The PCR product was purified, and digested by double enzymes, Bsp119I and XbaI, and meanwhile the expression vector pPICZαA was digested with these enzymes. The large fragment of the vector and the target gene fragment were recovered respectively, and the recovered fragments were ligated with T4 DNA ligase and transformed into Escherichia coli DH5α. After identification by colony PCR, the positive monoclonal bacterial solution was sequenced for verification.

2. Transformation of Pichia pastoris

The recombinant plasmids were first linearized with Sac I, and 1 ml of single-stranded DNA sample was boiled for 5 minutes and then rapidly cooled on ice. The samples were kept on ice. Competent yeast cells were centrifuged, and LiCl was removed with a pipette. 240 μl of 50% polyethylene glycol, 36 μl of 1 M LiCl, 25 μl of 2 mg/ml single-stranded DNAs, and plasmid DNAs (5 to 10 μg) in 50 μl of sterile water were sequentially added. Each tube was vortexed vigorously until the cell pellet was completely mixed (for about 1 minute). The test tubes were incubated at 30° C. for 30 minutes, and underwent a thermal shock in a water bath at 42° C. for 20 to 25 minutes. Cells were pelleted by centrifugation. The pellet was resuspended in 1 ml of YPD and incubated at 30° C. with oscillation. After 1 hour and 4 hours, 25 to 100 μl were inoculated on the YPD plates comprising an appropriate concentration of Zeocin™. The plates were incubated at 30° C. for 2 to 3 days.

10 single colonies were selected for enrichment culture, yeast chromosomal DNAs were extracted, and positive recombinant cells were detected by PCR. PCR identification was usually performed using pPICZαA universal primers. If the yeast expression vector pPICZαA was used as the template, a target fragment of about 588 bp can be amplified; and if pPICZαA-FTCD was used as the template, a target fragment with a target band size plus 588 bp can be amplified.

3. Enzyme Expression and Toxin Treatment

The screened positive yeast single colony (X33/pPICZαA-FTCD) and the negative yeast single colony (X33/pPICZαA) were respectively inoculated into 25 ml of BMGY medium, and cultured at 28° C. to 30° C. until OD600 was 2 to 6. The culture was centrifuged at room temperature, the supernatant was discarded, the cells were collected, the cells were resuspended in BMMY liquid medium to about OD600=1, transferred to a 500 ml Erlenmeyer flask, and cultured at 28° C. to 30° C., and methanol was added every 24 h to a final concentration of 0.5% to maintain induced expression. After 48 h of induction, the culture solution was aliquoted into 5 ml to 15 ml centrifuge tubes, and various trichothecenes were added to a final concentration of 25 μg/ml, the induction was continued for 48 h to 72 h, and the culture were collected for LC-HRMS analysis.

At the same time, after the positive yeast single colony (X33/pPICZαA-FTCD) and the negative yeast single colony (X33/pPICZαA) were induced for expressing proteins for 48 h, the culture was diluted with the medium at dilutions of 1, ⅕ and 1/20 (initial OD=0.01), and cultured on YPDA solid media with 400 μM DON and without DON for 5 days, and the growth was observed. The tolerances to DON were compared between transgenic yeast overexpressing active polypeptide and transgenic yeast with the blank vector.

4. LC-HRMS

The aliquoted samples were centrifuged, and the supernatant was discarded. The samples were quickly frozen in liquid nitrogen, a little quartz sand was added, and after grinding with a plastic grinding rod, 1.3 ml of pre-cooled 75% methanol aqueous solution (comprising 0.1% formic acid) was added. The mixture was vibrated for 10 s, sonicated for 30 min at room temperature, and the supernatant was taken and transferred to a new centrifuge tube. The supernatant was concentrated in vacuo to a dry powder. Before injection, the dry powder was resuspended with 100 μL of 20% acetonitrile solution, filtered through a 0.22 μm filter membrane, and transferred to an injection vial for LC-HRMS detection. The detection method was the same as above.

5. Experimental Results

5.1 LC-HRMS Results

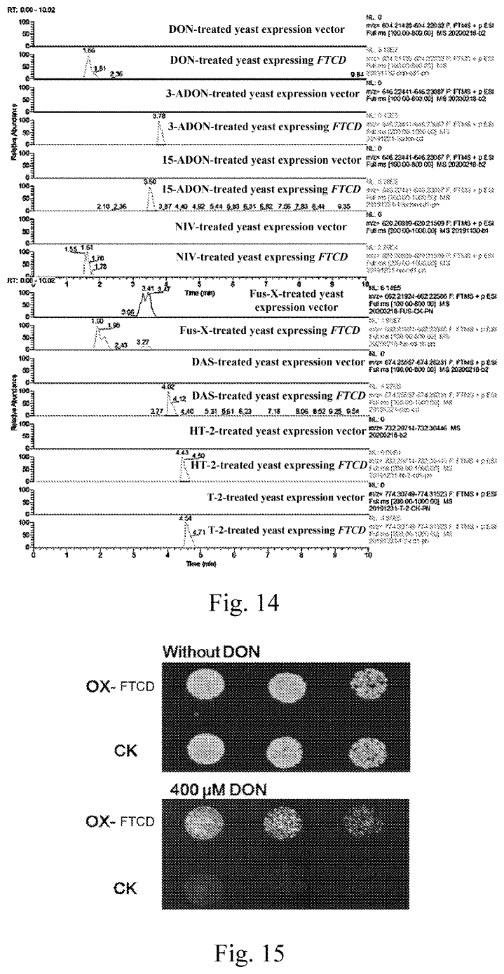

The LC-HRMS results were shown in FIG. 14. The DON-GSH adduct with an m/z of 604.21730 (corresponding to [M+H]⁺, Δ±5 ppm) was detected in positive ion mode by LC-HRMS (Full scan) from DON-treated yeast expressing the active polypeptide; the 3-ADON-GSH adduct with an m/z of 646.22764 (corresponding to [M+H]⁺, Δ±5 ppm) was detected from 3-ADON-treated yeast expressing the active polypeptide; the 15-ADON-GSH adduct with an m/z of 646.22764 (corresponding to [M+H]⁺, Δ±5 ppm) was detected from 15-ADON-treated yeast expressing the active polypeptide; the NIV-GSH adduct with an m/z of 620.21199 (corresponding to [M+H]⁺, Δ±5 ppm) was detected from NIV-treated yeast expressing active polypeptide; the DAS-GSH adduct with an m/z of 674.25894 (corresponding to [M+H]⁺, Δ±5 ppm) was detected from DAS-treated yeast expressing the active polypeptide; the “HT2-2H”-GSH adduct with an m/z of 730.28515 (corresponding to [M+H]⁺, Δ±5 ppm) was detected from HT-2-treated yeast expressing the active polypeptide; and the “T2-2H”-GSH adduct with an m/z of 772.29572 (corresponding to [M+H]⁺, Δ±5 ppm) was detected from T-2-treated yeast expressing FTCD. Meanwhile, no derivatives in the form of GSH adducts were detected in the corresponding controls.

The results of LC-HRMS detection showed that transfer of the de-epoxidase gene into Pichia pastoris can achieve efficient catalysis of conversion of trichothecene mycotoxins (comprising DON, 3-DON, 15-ADON, FUS-X, NIV, T-2, HT-2, and DAS) to glutathione adducts. Transgenic yeast had improved ability of toxin tolerance, demonstrating that FTCD can take a trichothecene mycotoxin as a substrate and catalyze it into the corresponding GSH adduct, thereby playing a role in detoxification in vivo.

5.2 Experimental Results of DON Tolerance of Transgenic Yeast

The growth viabilities of transgenic yeast overexpressing FTCD and transgenic yeast with the blank vector were compared on YPDA media with/without DON. A series of 1, ⅕, and 1/20-fold dilutions of yeast cultures with induced protein expression were added to yeast media (initial OD=0.01), and grown at 30° C. for 5 days, and the growth was observed. The results were shown in FIG. 15. It was found that the growth viability of transgenic yeast overexpressing FTCD on DON-containing media was significantly higher than that of transgenic yeast with the blank vector.

In the DON tolerance experiment of transgenic yeast, it was found that on the YPDA media comprising 400 μM DON, the growth viability of the transgenic yeast comprising FTCD was significantly higher than that of the transgenic yeast with the blank vector, further indicating that FTCD can be expressed in yeast and can catalyze the reaction between glutathione and a trichothecene such as DON for detoxification, thereby improving the tolerance of yeast to DON.

VI. Functional Analysis of the Gene of Homologous Sequences

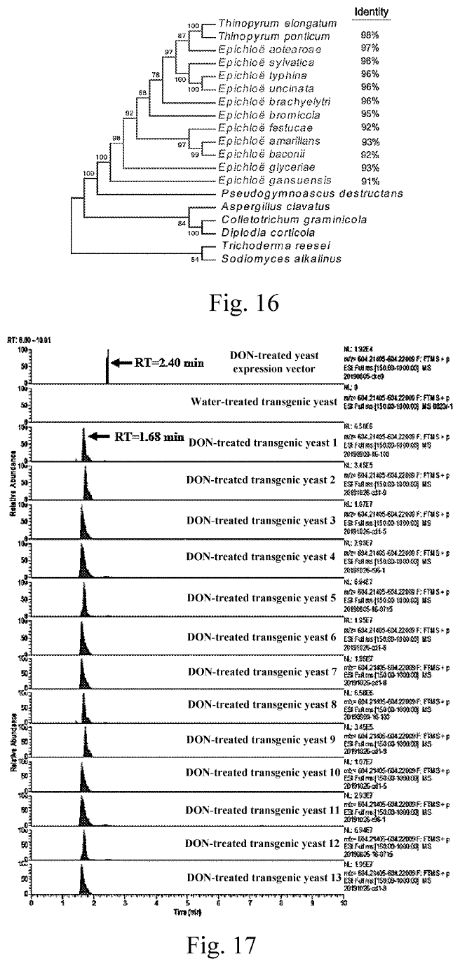

On the basis of the sequence (SEQ ID NO: 36) of the de-epoxidase gene derived from Thinopyrum, blastn alignment was performed by NCBI, and no annotated highly homologous gene was found under default parameters. However, according to the information that there were homologous genes among Epichloë sp., the inventor jointly searched the genome databases of other laboratories and obtained 11 sequences derived from this genus, as set forth in SEQ ID NOs: 60-70 respectively. As shown in FIG. 16, these sequences shared a sequence identity of 90% or more with the de-epoxidase gene of Thinopyrum ponticum. In addition, the inventor also isolated a gene from Thinopyrum elongatum with a sequence identity of 98% to the de-epoxidase gene of Thinopyrum ponticum, and its sequence was shown in SEQ ID NO: 37.

Using the same method as described above, these genes were transferred into yeast cells respectively, and expressed as corresponding proteins having amino acid sequences as set forth in SEQ ID Nos: 25-35 respectively. Analysis was performed using LC-HRMS. As shown in FIG. 17, other 12 homologous sequences were transferred into Pichia pastoris and treated with DON. LC-HRMS detection showed generation of DON-GSH. There was an independent and specific peak at RT=1.68 min, which was the GSH adduct at C-13 (by de-epoxidation). In extracted ion chromatograms of DON-treated transgenic yeast by LC-HRMS (Method 1), the DON-GSH adduct was detected in positive ion mode, with an m/z of 604.21730 (corresponding to [M+H]⁺, Δ±5 ppm).

On the basis of the above analysis, the inventor further analyzed the conservation between the proteins produced by these homologous genes, and obtained a polypeptide fragment having an amino acid sequence at positions 25-62, a polypeptide fragment having an amino acid sequence at positions 92-110, and a polypeptide fragment having an amino acid sequence at positions 144-184.

VII. Research on Mutation of FTCD

Using the Targeting Induced Local Lesions IN Genomes technology (TILLING technology), random mutation was performed on the de-epoxidase gene (with a sequence as set forth in SEQ ID NO: 36) derived from Thinopyrum ponticum to obtain 22 mutants of which the amino acid sequences were changed. The amino acid sequences of these mutants were shown in SEQ ID Nos: 3-24, respectively. After functional analysis, the original epoxy group-removing activity was retained to varying degrees in the 22 mutants. There were two termination mutations, terminating at amino acids 209 and 243, respectively, but the two termination mutations would not lead to complete loss of the enzyme's function. Therefore, it was suggested that the functional domain of this enzyme was mainly at the N-terminal.

After sequence homology analysis, two relatively conserved regions were found, i.e., a region at positions 92-104 and a region at positions 144-184. For the functions of these two conserved regions, it was speculated that they may be important regions related to catalytic activity. In the region between these two regions, there was a large variation among different species. Therefore, it was speculated that the region between these two conserved regions may be a linking region.

In order to verify the above speculation, the inventor designed a series of deletion mutants for verification based on the mutant materials obtained by screening the Tilling population in the early stage. Specifically, the SEQ ID NO: 36 sequence was taken as a template to design the corresponding specific primers comprising sequences homologous to the cloning vector, and the specific mutant types were as follows:

TABLE 1
Deletion mutant gene types

| | | | Position information of |
|---|---|---|---|
| | Position information | | corresponding amino acids |
| No. | of gene fragments | | of polypeptides |
| 1 | Full length | | 1-281 |
| 2 | 1-270 | bp | 1-90 |
| 3 | 1-570 | bp | 190 |
| 4 | 1-624 | bp | 1-208 |
| 5 | 1-726 | bp | 1-242 |
| 6 | 58-843 | bp | 20-281 |
| 7 | 118-843 | bp | 40-281 |
| 8 | 238-843 | bp | 80-281 |
| 9 | 283-843 | bp | 95-281 |
| 10 | 298-843 | bp | 100-281 |
| 11 | 448-843 | bp | 150-281 |
| 12 | 274-552 | bp | 91-184 |

| | | |
|---|---|---|
| 13 | 274 to 312 bp + a spacer | 92-104 + linker1 + 144-184 |
| | sequence + 430 to 552 bp | |
| 14 | 274 to 312 bp + a spacer | 92-104 + linker2 + 144-184 |
| | sequence + 430 to 552 bp | |
| 15 | 274 to 312 bp + a spacer | 92-104 + linker3 + 144-184 |
| | sequence + 430 to 552 bp | |
| 16 | 274 to 312 bp + a spacer | 92-104 + linker4 + 144-184 |
| | sequence + 430 to 552 bp | |
| 17 | 274 to 312 bp + an artificial | 92-104 + linker5 + 144-184 |
| | spacer sequence + 430 to 552 bp | |

Notes:
Linker 1 corresponds to the amino acid sequence at positions 105-142 in SEQ ID NO: 25;
Linker 2 corresponds to the amino acid sequence at positions 103-141 in SEQ ID NO: 26;
Linker 3 corresponds to the amino acid sequence at positions 107-148 in SEQ ID NO: 28;
Linker 4 corresponds to the amino acid sequence at positions 106-143 in SEQ ID NO: 30; and
Linker 5 is the artificial sequence GGGSGGSGG.

The specific experimental procedures were as follows:

1. The gene sequences corresponding to the above-mentioned deletion mutants were obtained by PCR, constructed into plasmid pET28a by the designed NcoI and BamHI restriction sites, transformed into Escherichia coli DH5α, identified by colony PCR and verified by sequencing. The correct recombinant expression vector plasmid was transformed into competent cells of Escherichia coli expression strain BL21 (DE3). The transformed cells were shaken at 37° C. for 4 h to induce protein expression with 0.5 mmol/L IPTG, and the bacterial cells were collected and resuspended in PBS. After ultrasonication, the supernatant was collected, and the supernatant was purified by a Ni column and a molecular sieve, and the purified protein was quantified by the BCA protein quantification method.

2. In Vitro Enzymatic Reaction

DON, 3-DON, and 15-ADON toxins (1 mg) were dissolved in freshly prepared GSH (30.7 mg, 100 μmol) in PBS buffer respectively, and the same amount of purified proteins were added based on the concentrations of proteins purified in vitro from several different FTCD deletion mutants, and incubated in a water bath at 25° C. for 24 h.

3. LC-HRMS (/MS) Analysis

The in vitro reaction solution was filtered through a 0.22 μm filter membrane, and transferred to an injection vial for LC-HRMS detection. PRM mode was used to quantify the relative abundance of toxins and their derivatives in a sample. The enzyme activity was calculated according to the amount of substrate conversion per unit time, and based on this result, the effect of different deletion mutations on protein activity was determined. DON, 3-DON, and 15-ADON toxins (1 mg) were dissolved in freshly prepared GSH (30.7 mg, 100 μmol) in PBS buffer respectively, and the same amount of purified proteins were added based on the concentrations of proteins purified in vitro from several different FTCD deletion mutants, and incubated in a water bath at 25° C. for 24 h. Samples were taken for LC-HRMS analysis, and the effect of different deletion mutations on enzyme activity was shown in Table 2.

TABLE 2
Experimental results of enzyme activity of different mutants

| | Amino acid | Specific enzyme activity (U/g) | | | |
|---|---|---|---|---|---|
| | sequence | | 3- | 15- | |
| No. | information | DON | ADON | ADON | Conclusion |
| 1 | Full length | 391.23 | 376.37 | 385.69 | |
| 2 | 1-90 | 0 | 0 | 0 | None |
| 3 | 1-190 | 321.33 | 305.32 | 320.29 | High |
| 4 | 1-208 | 321.33 | 317.01 | 329.36 | High |
| 5 | 1-242 | 316.32 | 329.10 | 334.05 | High |
| 6 | 20-281 | 302.33 | 301.27 | 319.11 | High |
| 7 | 40-281 | 305.36 | 304.84 | 307.45 | High, and |
| | | | | | decreased as |
| | | | | | compared with 3-5 |
| 8 | 80-281 | 294.63 | 282.45 | 283.12 | High, and |
| | | | | | decreased as |
| | | | | | compared with 3-5 |
| 9 | 95-281 | 20.45 | 33.22 | 28.28 | Very low |
| 10 | 100-281 | 22.01 | 27.38 | 19.19 | Very low |
| 11 | 150-281 | 0 | 0 | 0 | None |
| 12 | 92-184 | 250.21 | 245.10 | 249.17 | Lower than 6-7, |
| | | | | | but still having |
| | | | | | relatively high |
| | | | | | activity |
| 13 | 92-104 + | 208.78 | 212.67 | 195.37 | Slightly lower than |
| | linker1 + | | | | 11, but still having |
| | 144-184 | | | | relatively high |
| | | | | | activity |
| 14 | 92-104 + | 213.45 | 199.63 | 199.55 | Slightly lower than |
| | linker2 + | | | | 11, but still having |
| | 144-184 | | | | relatively high |
| | | | | | activity |
| 15 | 92-104 + | 211.97 | 204.83 | 214.58 | Slightly lower than |
| | linker3 + | | | | 11, but still having |
| | 144-184 | | | | relatively high |
| | | | | | activity |
| 16 | 92-104 + | 198.67 | 190.64 | 195.77 | Slightly lower than |
| | linker4 + | | | | 11, but still having |
| | 144-184 | | | | relatively high |
| | | | | | activity |
| 17 | 92-104 + | 169.37 | 176.48 | 169.97 | High |
| | linker5 + | | | | |
| | 144-184 | | | | |

It can be seen from Table 2 that the mutants with deletion of amino acids 1 to 90 had a little effect on the enzyme activity, while the deletion of the first 95 amino acids has a greater effect on the enzyme activity and led to greatly reduced enzyme activity, and the expressed protein was inactive if the first 150 amino acids were deleted. On the other hand, it was found that the sequence comprising the conserved region speculated by the present invention, i.e., the mutant comprising amino acids 92 to 184, had a high level of specific enzyme activity although the activity was affected. Hence, this was substantially in good agreement with what was speculated.

In addition, in the case of mutation of the speculated linking region, the sequence of this region of Thinopyrum ponticum was substituted with corresponding sequences of other genera respectively, and it was found that the activity remained substantially unchanged. Further substitution of this region with the artificially designed linker sequence GGGSGGSGG also had little effect on the enzyme activity. These results were substantially in good agreement with the predictions.

2. Mutation Analysis of Critical Amino Acids in Conserved Regions

On the basis of determining the critical regions of enzyme activity, the inventor further mutated the amino acids in the two conserved regions to expect to find critical amino acids.

Specifically, gene sequences having different mutation combinations were obtained by gene synthesis. These gene sequences were expressed in Escherichia coli and purified. The resulting mutant polypeptides were used for the in vitro enzymatic reaction, and the enzyme activity was analyzed by LC-HRMS (/MS). The results were shown in Table 3.

TABLE 3
Experiment on the effect of amino acid mutations in conserved regions on the enzyme activity

| | Mutation types of functional | Specific enzyme activity (U/g) | |
|---|---|---|---|
| No. | domains | DON | Description |
| 1 | Wild type | 269.43 | Corresponding to positions 92 to 184 of |
| | | | SEQ ID NO: 1 |
| 2 | S94G | 203.79 | High |
| 3 | F95L | 152.39 | Slightly low as compared with other |
| | | | mutants having a single mutation, but still |
| | | | maintaining most of the activity |
| 4 | A98V | 8.97 | Very low activity |
| 5 | A99V | 15.81 | Very low activity |
| 6 | Y100H | 102.93 | Slightly low as compared with other |
| | | | mutants having a single mutation, but still |
| | | | maintaining about half of the activity |
| 7 | L101V | 26.47 | Very low activity |
| 8 | T104S | 8.73 | Very low activity |
| 9 | D145E | 36.73 | Very low activity |
| 10 | N150Y | 29.33 | Very low activity |
| 11 | S151G | 196.71 | High |
| 12 | V153A | 35.65 | Very low activity |
| 13 | D154E | 28.99 | Very low activity |
| 14 | A155V | 206.55 | High |
| 15 | A156V | 32.14 | Very low activity |
| 16 | F157C | 18.63 | Very low activity |
| 17 | T158Q | 185.76 | High |
| 18 | T158N | 194.28 | High |
| 19 | A159T | 27.45 | Very low activity |
| 20 | H160R | 26.66 | Very low activity |
| 21 | V161A | 40.25 | Very low activity |
| 22 | G162S | 217.76 | High |
| 23 | L163V | 167.95 | Slightly low as compared with other |
| | | | mutants having a single mutation, but still |
| | | | maintaining most of the activity |
| 24 | M164L | 19.63 | Very low activity |
| 25 | V165L | 165.44 | Slightly low as compared with other |
| | | | mutants having a single mutation, but still |
| | | | maintaining most of the activity |
| 26 | P169R | 38.21 | Very low activity |
| 27 | L170V | 19.67 | Very low activity |
| 28 | P172V | 10.34 | Very low activity |
| 29 | T174S | 204.27 | High |
| 30 | A175V | 183.34 | High |
| 31 | D176E | 24.98 | Very low activity |
| 32 | T178I | 100.49 | Slightly low as compared with other |
| | | | mutants having a single mutation, but still |
| | | | maintaining about half of the activity |
| 33 | K179E | 27.65 | Very low activity |
| 34 | R184P | 18.96 | Very low activity |
| 35 | S94G + A175V | 148.78 | High, maintaining most of the activity |
| 36 | F95L + A175V | 155.21 | High, maintaining most of the activity |
| 37 | S94G + Y100H | 112.16 | Slightly low, but still maintaining half of |
| | | | the activity |
| 38 | S94G + S151G | 144.51 | High, maintaining most of the activity |
| 39 | S94G + A155V | 162.43 | High, maintaining most of the activity |
| 40 | S94G + L163V | 145.48 | High, maintaining most of the activity |
| 41 | S94G + T174S | 155.58 | High, maintaining most of the activity |
| 42 | S94G + T178I | 114.29 | Maintaining half of the activity |
| 43 | F95L + L163V | 104.31 | Slightly low, but still maintaining half of |
| | | | the activity |
| 44 | F95L + T158Q | 151.57 | High, maintaining most of the activity |
| 45 | F95L + T174S | 145.07 | High, maintaining most of the activity |
| 46 | F95L + V165L | 102.30 | Slightly low, but still maintaining half of |
| | | | the activity |
| 47 | F95L + T178I | 70.09 | Relatively low, with less than half of the |
| | | | activity of the wild type |
| 48 | F95L + Y100H | 81.42 | Relatively low, with less than half of the |
| | | | activity of the wild type |
| 49 | F95L + S151G | 157.81 | High, maintaining most of the activity |
| 50 | Y100H + T158Q | 132.45 | High, maintaining most of the activity |
| 51 | Y100H + A175V | 127.94 | High, maintaining most of the activity |
| 52 | Y100H + L163V | 97.93 | Relatively low, with less than half of the |
| | | | activity of the wild type |
| 53 | Y100H + G162S | 113.19 | Slightly low, but still maintaining half of |
| | | | the activity |
| 54 | Y100H + T178I | 58.11 | Very low |
| 55 | Y100H + A155V | 102.65 | Slightly low, but still maintaining half of |
| | | | the activity |
| 56 | V165L + T174S | 142.16 | High, maintaining most of the activity |
| 57 | V165L + T178I | 90.56 | Relatively low, with less than half of the |
| | | | activity of the wild type |
| 58 | S151G + T178I | 150.27 | High |
| 59 | S94G + T158Q | 161.94 | High |
| 60 | A155V + G162S | 173.73 | High |
| 61 | A155V + V165L | 149.12 | High, maintaining most of the activity |
| 62 | A155V + T174S | 159.77 | High |
| 63 | A155V + A175V | 156.82 | High |
| 64 | A155V + T178I | 163.51 | High |
| 65 | S151G + T158N | 154.05 | High |
| 66 | S151G + L163V | 148.53 | High, maintaining most of the activity |
| 67 | S151G + V165L | 149.33 | High, maintaining most of the activity |
| 68 | S151G + A175V | 166.06 | High |
| 69 | L163V + V165L | 140.65 | High, maintaining most of the activity |
| 70 | L163V + T174S | 156.79 | High, maintaining most of the activity |
| 71 | L163V + A175V | 159.05 | High, maintaining most of the activity |
| 72 | L163V + T178I | 67.94 | Relatively low, with less than half of the |
| | | | activity of the wild type |
| 73 | S94G + T158Q + G162S | 149.64 | High, maintaining most of the activity |
| 74 | S94G + T158N + G162S | 134.55 | High, maintaining most of the activity |
| 75 | S94G + F95L + Y100H | 100.96 | Relatively low, with less than half of the |
| | | | activity of the wild type |
| 76 | F95L + Y100H + G162S | 95.95 | Relatively low, with less than half of the |
| | | | activity of the wild type |
| 77 | S94G + Y100H + S151G | 86.79 | Relatively low, with less than half of the |
| | | | activity of the wild type |
| 78 | S94G + S151G + A155V | 150.37 | High, maintaining most of the activity |
| 79 | S94G + A155V + T174S | 163.93 | High, maintaining most of the activity |
| 80 | S94G + L163V + A175V | 142.37 | High, maintaining most of the activity |
| 81 | S94G + T174S + A175V | 146.45 | High, maintaining most of the activity |
| 82 | S94G + V165L + T178I | 152.06 | High, maintaining most of the activity |
| 83 | F95L + Y100H + A175V | 92.06 | Relatively low, with less than half of the |
| | | | activity of the wild type |
| 84 | F95L + T158Q + G162S | 158.26 | High, maintaining most of the activity |
| 85 | F95L + T174S + T178I | 86.21 | Relatively low, with less than half of the |
| | | | activity of the wild type |
| 86 | S94G + V165L + T174S | 148.93 | High, maintaining most of the activity |
| 87 | F95L + G162S + V165L | 174.58 | Slightly low, but still maintaining half of |
| | | | the activity |
| 88 | F95L + Y100H + S151G | 201.97 | Relatively low, with less than half of the |
| | | | activity of the wild type |
| 89 | F95L + S151G + T158Q | 183.79 | High, maintaining most of the activity |
| 90 | F95L + L163V + V165L | 196.22 | Relatively low, with less than half of the |
| | | | activity of the wild type |
| 91 | F95L + G162S + L163V | 122.97 | Slightly low, but still maintaining half of |
| | | | the activity |
| 92 | S94G + Y100H + T158Q | 111.39 | Slightly low, but still maintaining half of |
| | | | the activity |
| 93 | F95L + S151G + T158N | 133.72 | High, maintaining most of the activity |
| 94 | Y100H + T158Q + G162S | 116.46 | Slightly low, but still maintaining half of |
| | | | the activity |
| 95 | Y100H + T174S + A175V | 107.66 | Slightly low, but still maintaining half of |
| | | | the activity |
| 96 | Y100H + A175V + T178I | 49.34 | Relatively low, with less than half of the |
| | | | activity of the wild type |
| 97 | Y100H + A155V + L163V | 96.46 | Relatively low, with less than half of the |
| | | | activity of the wild type |
| 98 | S151G + T158N + G162S | 149.59 | High, maintaining most of the activity |
| 99 | S151G + L163V + A175V | 168.97 | High, maintaining most of the activity |
| 100 | S151G + V165L + T178I | 73.56 | Relatively low, with less than half of the |
| | | | activity of the wild type |
| 101 | S151G + A175V + T178I | 57.24 | Very low |
| 102 | L163V + V165L + T174S | 104.25 | Slightly low, but still maintaining half of |
| | | | the activity |
| 103 | L163V + T174S + A175V | 137.13 | High, maintaining most of the activity |
| 104 | S94G + A155V + T158Q | 149.59 | High, maintaining most of the activity |
| 105 | S94G + L163V + V165L | 109.29 | Slightly low, but still maintaining half of |
| | | | the activity |
| 106 | S94G + T174S + T178I | 114.18 | Slightly low, but still maintaining half of |
| | | | the activity |
| 107 | S94G + F95L + L163V | 93.66 | Relatively low, with less than half of the |
| | | | activity of the wild type |
| 108 | F95L + A155V + T178I | 78.62 | Relatively low, with less than half of the |
| | | | activity of the wild type |
| 109 | F95L + T158Q + L163V | 127.47 | Slightly low, but still maintaining half of |
| | | | the activity |
| 110 | F95L + T174S + A175V | 144.93 | High, maintaining most of the activity |
| 111 | F95L + V165L + T174S | 104.66 | Slightly low, but still maintaining half of |
| | | | the activity |
| 112 | Y100H + A155V + T178I | 59.33 | Relatively low, with less than half of the |
| | | | activity of the wild type |
| 113 | F95L + Y100H + L163V | 62.31 | Very low |
| 114 | Y100H + S151G + A155V | 139.45 | High, maintaining most of the activity |
| 115 | A155V + L163V + T174S | 146.42 | High, maintaining most of the activity |
| 116 | A155V + T174S + T178I | 87.87 | Relatively low, with less than half of the |
| | | | activity of the wild type |
| 117 | Y100H + A155V + V165L | 93.59 | Relatively low, with less than half of the |
| | | | activity of the wild type |
| 118 | S151G + T158N + V165L | 142.27 | High, maintaining most of the activity |
| 119 | S151G + L163V + T174S | 136.38 | High, maintaining most of the activity |
| 120 | S151G + V165L + A175V | 158.62 | High, maintaining most of the activity |
| 121 | V165L + T174S + A175V | 158.35 | High, maintaining most of the activity |
| 122 | S151G + V165L + T178I | 86.54 | Relatively low, with less than half of the |
| | | | activity of the wild type |
| 123 | S151G + A175V + T178I | 72.57 | Relatively low, with less than half of the |
| | | | activity of the wild type |
| 124 | S94G + F95L + Y100H + L163V | 55.92 | Very low |
| 125 | S94G + S151G + A155V + T174S | 130.29 | High, maintaining most of the activity |
| 126 | S94G + F95L + Y100H + T158Q | 89.79 | Relatively low, with less than half of the |
| | | | activity of the wild type |
| 127 | S94G + F95L + Y100H + S151G | 75.05 | Relatively low, with less than half of the |
| | | | activity of the wild type |
| 128 | S94G + F95L + Y100H + T174S | 76.21 | Relatively low, with less than half of the |
| | | | activity of the wild type |
| 129 | S94G + F95L + S151G + A155V | 136.63 | High, maintaining most of the activity |
| 130 | S94G + F95L + S151G + T158N | 152.75 | High, maintaining most of the activity |
| 131 | S94G + F95L + S151G + T174S | 135.95 | High, maintaining most of the activity |
| 132 | S94G + F95L + S151G + L163V | 106.16 | Slightly low, but still maintaining half of |
| | | | the activity |
| 133 | S94G + F95L + A155V + T158Q | 151.87 | High, maintaining most of the activity |
| 134 | S94G + F95L + A155V + V165L | 112.11 | Slightly low, but still maintaining half of |
| | | | the activity |
| 135 | F95L + Y100H + S151G + T158Q | 61.99 | Relatively low, with less than half of the |
| | | | activity of the wild type |
| 136 | S94G + Y100H + T158Q + T174S | 70.93 | Relatively low, with less than half of the |
| | | | activity of the wild type |
| 137 | F95L + Y100H + G162S + T174S | 71.33 | Relatively low, with less than half of the |
| | | | activity of the wild type |
| 138 | S94G + F95L + Y100H + T178I | 50.39 | Relatively low, with less than half of the |
| | | | activity of the wild type |
| 139 | S94G + Y100H + S151G + T158Q | 57.60 | Relatively low, with less than half of the |
| | | | activity of the wild type |
| 140 | F95L + Y100H + S151G + T174S | 64.47 | Relatively low, with less than half of the |
| | | | activity of the wild type |
| 141 | S94G + Y100H + T158N + L163V | 69.73 | Relatively low, with less than half of the |
| | | | activity of the wild type |
| 142 | F95L + Y100H + A155V + T174S | 70.51 | Relatively low, with less than half of the |
| | | | activity of the wild type |
| 143 | F95L + Y100H + T158N + T174S | 52.88 | Relatively low, with less than half of the |
| | | | activity of the wild type |
| 144 | S94G + F95L + S151G + G162S | 132.06 | High, maintaining most of the activity |
| 145 | F95L + S151G + T158Q + G162S | 142.63 | High, maintaining most of the activity |
| 146 | F95L + Y100H + T158Q + T178I | 39.33 | Very low |
| 147 | S94G + Y100H + T158Q + G162S | 59.90 | Relatively low, with less than half of the |
| | | | activity of the wild type |
| 148 | F95L + Y100H + T158Q + A175V | 60.04 | Relatively low, with less than half of the |
| | | | activity of the wild type |
| 149 | F95L + A155V + G162S + T178I | 67.25 | Relatively low, with less than half of the |
| | | | activity of the wild type |
| 150 | F95L + T158N + G162S + T174S | 145.33 | High, maintaining most of the activity |
| 151 | F95L + T158Q + G162S + A175V | 148.73 | High, maintaining most of the activity |
| 152 | F95L + T158Q + V165L + T174S | 99.76 | Slightly low, but still maintaining half of |
| | | | the activity |
| 153 | Y100H + S151G + G162S + T178I | 38.73 | Relatively low, with less than half of the |
| | | | activity of the wild type |
| 154 | F95L + Y100H + A155V + T158Q | 77.29 | Relatively low, with less than half of the |
| | | | activity of the wild type |
| 155 | S94G + A155V + T158Q + G162S | 149.04 | High, maintaining most of the activity |
| 156 | S94G + S151G + T158Q + T174S | 150.51 | High, maintaining most of the activity |
| 157 | S94G + A155V + T158N + G162S | 56.49 | Relatively low, with less than half of the |
| | | | activity of the wild type |
| 158 | S151G + A155V + G162S + T178I | 49.46 | Very low |
| 159 | S94G + T158Q + G162S + L163V | 178.68 | High, maintaining most of the activity |
| 160 | F95L + A155V + T158N + T174S | 113.61 | High, maintaining most of the activity |
| 161 | Y100H + G162S + T174S + T178I | 71.33 | Relatively low, with less than half of the |
| | | | activity of the wild type |
| 162 | S94G + T158N + V165L + T174S | 141.18 | High, maintaining most of the activity |
| 163 | S151G + T158Q + T174S + T178I | 56.45 | Very low |
| 164 | Y100H + S151G + A155V + L163V | 52.39 | Relatively low, with less than half of the |
| | | | activity of the wild type |
| 165 | S151G + A155V + T158Q + L163V | 159.93 | High, maintaining most of the activity |
| 166 | A155V + T158Q + G162S + A175V | 133.41 | High, maintaining most of the activity |
| 167 | A155V + T158Q + G162S + T174S | 130.05 | High, maintaining most of the activity |
| 168 | T158N + G162S + L163V + T178I | 29.74 | Very low |
| 169 | A155V + G162S + L163V + V165L | 102.12 | Slightly low, but still maintaining half of |
| | | | the activity |
| 170 | S151G + A155V + T174S + T178I | 54.79 | Very low |
| 171 | S151G + T158Q + V165L + A175V | 145.16 | High, maintaining most of the activity |
| 172 | S151G + Y100H + L163V + A175V | 59.04 | Relatively low, with less than half of the |
| | | | activity of the wild type |
| 173 | S151G + A155V + A175V + T178I | 137.81 | High, maintaining most of the activity |
| 174 | S151G + T158N + V165L + T174S | 173.16 | High, maintaining most of the activity |
| 175 | Y100H + S151G + A155V + T158Q | 55.38 | Very low |
| 176 | Y100H + A155V + T158Q + L163V | 54.68 | Relatively low, with less than half of the |
| | | | activity of the wild type |
| 177 | Y100H + T158Q + G162S + L163V | 64.47 | Relatively low, with less than half of the |
| | | | activity of the wild type |
| 178 | Y100H + T158N + V165L + A175V | 68.57 | Relatively low, with less than half of the |
| | | | activity of the wild type |
| 179 | Y100H + A155V + T174S + T178I | 38.45 | Relatively low, with less than half of the |
| | | | activity of the wild type |
| 180 | Y100H + T158N + T174S + A175V | 72.46 | Relatively low, with less than half of the |
| | | | activity of the wild type |
| 181 | Y100H + A155V + V165L + T178I | 60.78 | Relatively low, with less than half of the |
| | | | activity of the wild type |
| 182 | Y100H + T158Q + T174S + T178I | 63.48 | Relatively low, with less than half of the |
| | | | activity of the wild type |
| 183 | Y100H + A155V + T158N + G162S | 65.71 | Relatively low, with less than half of the |
| | | | activity of the wild type |
| 184 | Y100H + T158N + L163V + V165L | 41.91 | Very low |
| 185 | S151G + A155V + T158Q + V165L | 130.59 | High, maintaining most of the activity |
| 186 | S151G + T158Q + G162S + L163V | 131.98 | High, maintaining most of the activity |
| 187 | S151G + A155V + L163V + T174S | 144.33 | High, maintaining most of the activity |
| 188 | S151G + G162S + T174S + T178I | 61.25 | Relatively low, with less than half of the |
| | | | activity of the wild type |
| 189 | S151G + T158N + V165L + A175V | 138.02 | High, maintaining most of the activity |
| 190 | A155V + T158Q + G162S + L163V | 122.29 | High, maintaining most of the activity |
| 191 | A155V + T158N + V165L + A175V | 114.30 | High, maintaining most of the activity |
| 192 | A155V + G162S + L163V + T178I | 53.78 | Very low |
| 193 | A155V + L163V + A175V + T178I | 52.07 | Very low |
| 194 | A155V + T158N + T174S + T178I | 81.15 | Relatively low, with less than half of the |
| | | | activity of the wild type |
| 195 | T158Q + G162S + L163V + T174S | 129.29 | High, maintaining most of the activity |
| 196 | T158Q + V165L + A175V + T178I | 67.25 | Relatively low, with less than half of the |
| | | | activity of the wild type |
| 197 | T158Q + G162S + T174S + T178I | 50.55 | Relatively low, with less than half of the |
| | | | activity of the wild type |
| 198 | T158Q + L163V + T174S + A175V | 127.24 | High, maintaining most of the activity |
| 199 | T158N + L163V + T174S + T178I | 100.35 | Relatively low, with less than half of the |
| | | | activity of the wild type |
| 200 | T158N + G162S + V165L + A175V | 119.25 | High, maintaining most of the activity |
| 201 | S94G + F95L + Y100H + T158Q + | 41.91 | Very low |
| | L163V | | |
| 202 | S94G + F95L + T158N + G162S + | 101.19 | Slightly low, but still maintaining half of |
| | T174S | | the activity |
| 203 | F95L + A155V + G162S + V165L + | 37.24 | Very low |
| | T178I | | |
| 204 | F95L + Y100H + T158Q + G162S + | 72.29 | Relatively low, with less than half of the |
| | T174S | | activity of the wild type |
| 205 | F95L + Y100H + T158Q + L163V + | 21.06 | Very low |
| | A175V | | |
| 206 | S94G + A155V + G162S + L163V + | 73.64 | Relatively low, with less than half of the |
| | T174S + T178I | | activity of the wild type |
| 207 | F95L + Y100H + A155V + G162S + | 63.9 | Relatively low, with less than half of the |
| | L163V + T178I | | activity of the wild type |
| 208 | F95L + Y100H + S151G + T158Q + | 81.3 | Relatively low, with less than half of the |
| | G162S + T174S + T178I | | activity of the wild type |
| 209 | S94G + Y100H + A155V + T158N + | 15.87 | Very low |
| | G162S + L163V + V165L + A175V | | |
| 210 | F95L + Y100H + S151G + T158N + | 20.34 | Very low activity |
| | G162S + T174S + T178I | | |
| 211 | S94G + Y100H + A155V + T158Q + | 48.67 | Very low |
| | G162S + L163V + V165L + A175V | | |
| 212 | G162S + L163V + V165L + T174S + | 103.33 | Slightly low, but still maintaining half of |
| | A175V | | the activity |
| 213 | T158N + G162S + V165L + A175V + | 20.63 | Very low |
| | T178I | | |
| 214 | T158Q + T174S + A175V + T178I | 101.78 | Slightly low, but still maintaining half of |
| | | | the activity |
| 215 | G162S + L163V + V165L + T178I | 13.37 | Very low |
| 216 | L163V + V165L + T174S + A175V | 97.33 | Slightly low, but still maintaining half of |
| | | | the activity |
| 217 | L163V + T174S + A175V + T178I | 60.85 | Relatively low, with less than half of the |
| | | | activity of the wild type |
| 218 | T158N + T174S + A175V + T178I | 56.64 | Relatively low, with less than half of the |
| | | | activity of the wild type |

There were some variable sites in the conserved sequence of FTCD, wherein after the amino acids at positions 94, 95, 100, 151, 155, 158, 162, 163, 165, 174, 175 and 178 were changed, FTCD can still maintain a certain activity. Among these variable sites, amino acid changes at different sites had different effects on the activity of FTCD, wherein when the amino acids at positions 100 and 178 were changed, the activity of FTCD was greatly affected, and the activity can be reduced by about 60%. After other variable sites were changed, the activity of FTCD can remain 50% or more.

VIII. Study on the Expression of the De-Epoxidase Gene of Epichloë in Different Engineered Microorganisms

The strains, vectors or plasmids used in this example were all preserved in our laboratory unless otherwise stated. The plasmid pMD19-T-FTCD comprises a de-epoxidase gene derived from Epichloë, having a sequence as set forth in SEQ ID NO: 68.

8.1 Efficient Secretion and Expression of the De-Epoxidase Gene of Epichloë in Bacillus subtilis

8.1.1 Preparation of Competent Cells

The bacterial solution of Bacillus subtilis was spread on an LB solid medium, and cultured at 37° C. overnight. Single colonies were picked and inoculated into 5 ml of GMI medium. The culture was shaken overnight at 30° C. and 130 r/min. 2 ml of overnight culture was pipetted into 18 ml of GMI medium, and cultured at 37° C. and 250 r/min for 3.5 h. The above culture was transferred into 10 ml of GMII medium in the same proportion, and cultured at 37° C. and 130 r/min for 1.5 h. 1 mL of the above-mentioned second-passage culture was taken and centrifuged at 5,000 r/min at room temperature for 5 min, and the bacterial pellet was resuspended with 1/10 volume of the supernatant, obtaining the competent cells of Bacillus subtilis.

8.1.2 Construction of Recombinant Expression Vector pHT43-FTCD

The primers with BamHI and SamI restriction sites were designed according to the sequence of expression vector pHT43, and the primer sequences were as follows (underlined sequences indicate the restriction sites):

Forward primer:
5′-CGTAGGATCCATGGCCACCCCCACCTCCAC-3′
Reverse primer:
5′-CTGCCCCGGGCTTCACCTCGGCATACTTGTC-3′

PCR amplification was performed using plasmid pMD19-T-FTCD as a template. The amplification product was detected by 1% agarose gel electrophoresis, and a target fragment was recovered by cutting the gel; the target fragment and pHT43 vector were digested by double enzymes, BamHI and SamI, respectively, followed by gel recovery and ligation with T4 ligase; the ligation product was mixed with competent cells of Bacillus subtilis to a final concentration of 1 μg/mL. After mixing well, the mixture was placed in a water bath at 37° C. and left to stand for 30 to 60 min, and shaken at 37° C. and 220 r/min for 4 h. After shaking, the mixture of the recombinant plasmid and the competent cells were spread on a chloramphenicol-resistant medium and cultured overnight at 37° C.; single colonies were picked and identified by colony PCR and double digestion to obtain a target gene of about 900 bp and a pHT43 vector backbone of about 8,000 bp. Further sequencing was performed to verify that the sequence and the reading frame of the recombinant expression vector pHT43-FTCD were correct.

8.1.3 Induced Expression of Polypeptides

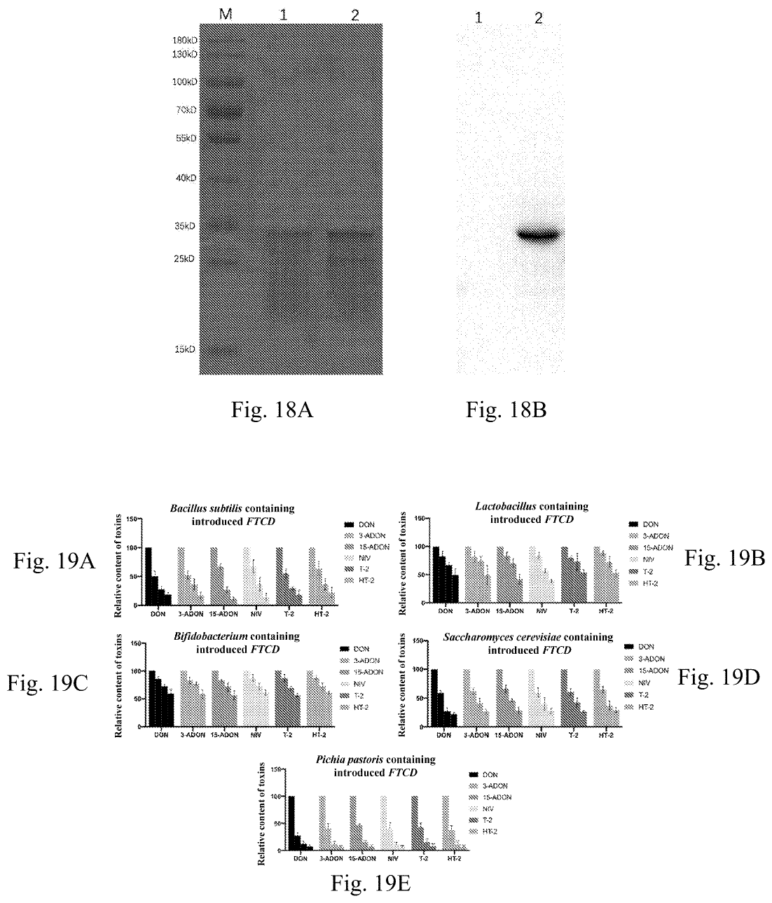

The target protein in Bacillus subtilis was mainly secreted into the medium in a soluble state. After the positive bacteria comprising the recombinant plasmids were subjected to expanded culture, they were shaken in LB broth media until the OD600 of the bacteria was 0.6 to 0.8. 1 mL of the culture was taken out and centrifuged at room temperature for 2 min, the supernatant was discarded, and the bacterial pellet was resuspended in 100 μl of 1× loading buffer. IPTG was added to the remaining culture to a final concentration of 0.5 mM, and the fusion protein was induced to express by shaking at 37° C. and 220 r/min for 8 h. Samples were taken at 4 h and 8 h, respectively, and centrifuged to obtain a supernatant for SDS-PAGE and western blot detection. The results were shown in FIGS. 18A and 18B, indicating that soluble protein was obtained in the medium with a molecular weight of about 32 kDa, and the expression level after 8 h of induction was higher. Western Blot was performed using an His-tagged antibody, and a protein band with a molecular weight of about 32 kDa appeared, while no immunoreactive band was found in the control group.

8.1.4. The Reaction of Removing an Epoxy Group of Vomitoxin Catalyzed by the Fermentation Supernatant

8.1.4.1 Experimental Methods:

8.1.4.1.1 In vitro catalysis of reaction by the fermentation supernatant:

DON, 3-DON, 15-ADON, NIV, DAS, HT-2, and T-2 toxins (1 mg) were dissolved in freshly prepared GSH (30.7 mg, 100 μmol) in PBS buffer respectively, and the fermentation supernatant that was concentrated 10 times using an ultrafiltration membrane was added, and incubated in a water bath at 20° C. for 24 h.

8.1.4.1.2 LC-HRMS (/MS) Analysis

The in vitro reaction solution was filtered through a 0.22 μm filter membrane, and transferred to an injection vial for LC-HRMS detection.

Chromatography was performed on a reverse phase XBridge C18, with an inner diameter of 150><2.1 mm, and a particle size of 3.5 μm (Waters, Dublin, Ireland), at a column temperature of 35° C. The flow rate was 300 μL min⁻¹, and the injection volume was 3 μL. Mobile phase: A: 0.1% aqueous acetic acid, B: acetonitrile; elution gradient: A=90% at 0 to 0.2 min; A gradually decreased to 10% at 0.2 to 6 min; A=10% at 6 to 8 min; A gradually increased to 90% at 8.1 min; and A=90% at 8.1 to 10 min.

Xcalibur 2.1.0 (Thermo Fisher Scientific, San Jose, Calif., USA) were used for analysis of data of LC-HRMS (/MS). Extracted ion chromatograms (EICs) of toxins and their derivatives were investigated using the extracted chromatographic peak shape, retention time (±0.2 min) and mass (±5 ppm) of the bioconversion products. According to secondary spectra and basic structures of the substances, the neutral loss was analyzed, and chemical structures were inferred.

8.1.4.2 Experimental Results

The active protein contained in the fermentation supernatant produced by the Bacillus subtilis expression system can efficiently catalyze trichothecene mycotoxins (comprising DON, 3DON, 15ADON, FUS-X, NIV, T2, H-T2, DAS, and the like) into glutathione adducts in vitro, and it can be seen from the secondary spectrum that the formation of the adducts destroyed the epoxy ring structure playing a major role in the toxicity of trichothecenes, which can greatly reduce the toxicity of the toxins.

8.2 Efficient Expression of the De-Epoxidase Gene of Epichloë in Lactobacillus

Unless specifically stated otherwise, the contents in this step is the same as in 8.1.

8.2.1. Preparation of competent cells

A bacterial solution of Lactobacillus MG1363 in glycerin was spread on an GM17 solid medium and cultured at 30° C. for 24 h; single colonies were picked and inoculated into 3 ml of GSGM17B medium. The bacteria were cultured by standing at 30° C. for 12 h; 2 ml of overnight culture was pipetted into 100 ml of GSGM17B medium, and cultured at 30° C. until OD600 was 0.3 to 0.5; the culture was centrifuged at 4° C. and 6,000 rpm for 20 min using a centrifuge to collect bacteria; the bacteria were resuspended with 100 ml of pre-cooled EPB, and centrifuged at 4° C. and 6,000 rpm for 20 min, and the supernatant was discarded; the bacteria were resuspended with 100 ml of pre-cooled EPB+EDTA, placed in an ice bath for 15 min, and centrifuged at 4° C. and 6,000 rpm for 20 min, and the supernatant was discarded; the bacteria were resuspended again with 25 ml of pre-cooled EPB, and centrifuged at 4° C. and 6,000 rpm for 20 min, and the supernatant was discarded; the bacteria were resuspended with 1 ml of pre-chilled EPB, aliquoted on ice, and stored at −80° C.

8.2.2. Construction of Recombinant Expression Vector pMG36e-FTCD

The primers with SamI and HindIII restriction sites were designed according to the sequence of expression vector pMG36e, and the primer sequences were as follows (underlined sequences indicate the restriction sites):

Forward primer:
5′-AAGCTTCTAGAAATCCACCCATCGTCATCACC-3′
Reverse primer:
5′-CCCGGGTCTTCACCTCGGCATACTTGTC-3′

PCR amplification was performed using plasmid pMD19-T-FTCD as a template. The amplification product was detected by 1% agarose gel electrophoresis, and a target fragment was recovered by cutting the gel; the target fragment and pMG36e vector were digested by double enzymes, SamI and HindIII, respectively, followed by gel recovery and ligation at 16° C. overnight.

8.2.3. Construction of Recombinant Lactobacillus by Electrotransformation

The recombinant plasmid pMG36e-FTCD was introduced into competent cells of Lactobacillus MG1363 by electrotransformation to obtain recombinant Lactobacillus. An empty vector was transformed into Lactobacillus by the same method as a control. Single colonies were randomly picked from the transformation plate and identified by colony PCR and double digestion to obtain a target gene of about 900 bp and a pMG36e vector backbone of about 3,600 bp. Further sequencing was performed to verify that the sequence and the reading frame of the recombinant expression vector pMG36e-FTCD were correct.

8.2.4. FTCD Expression in Lactobacillus

The target protein in Bacillus subtilis was mainly secreted into the medium in a soluble state. After the positive bacteria comprising the recombinant plasmids were subjected to expanded culture, they were shaken in GSGM17B broth media until the OD600 of the bacteria was 0.6 to 0.8. 1 mL of the culture was taken out and centrifuged at room temperature for 2 min, the supernatant was discarded, and the bacterial pellet was lysed in 100 μl of 1× loading buffer. The solution was centrifuged to obtain a supernatant for SDS-PAGE and western blot detection. The results showed that a soluble protein was obtained in the medium.

8.3 Efficient Expression of the De-Epoxidase Gene of Epichloë in Bifidobacterium (Cultured Under Anaerobic Conditions)

Unless specifically stated otherwise, the contents in this step is the same as in 8.1.

8.3.1. Construction of a Bifidobacterium Secretory Expression Vector

According to the study results of Xun Anying, et al., a secretory expression vector was constructed. pBAD-gIIIA was used as a template to amplified a partial sequence, comprising the promoter sequence of the arabinose operon (PBAD) and the positive and negative regulator gene (araC) sequence of the promoter, without the signal peptide sequence. The amplification primer sequences were as follows (the forward primer had an Mph1103I restriction site at the 5′ terminal):

Forward primer:
5′-GGTGGTATGCATATGCTACTCCGTCAAGCCGT-3′;
reverse primer:
5′-GTTAATTCCTCCTGTTAGCC-3′

The endogenous arabinosidase secretory signal peptide sequence of Bifidobacterium was amplified by PCR using the genomic DNA of Bifidobacterium as a template.

Forward primer:
5′-GGCTAACAGGAGGAATTAACCATGAATTATTTACGACAAAAA-3′;
reverse primer:
5′-GTTGTTCCATGGAAGACTCCGCAAAGACCGGCATTGGCC-3′

The above-mentioned two fragments were ligated, the plasmid pBAD-gIII and the ligated fragment were digested with Mph1103I and NcoI, and then recovered and ligated to construct a plasmid, which was verified by sequencing; and the natural Bifidobacterium plasmid polymerase gene (BPP gene) was cloned, digested and ligated to the plasmid to construct the plasmid pBBADs.

8.3.2. Construction of pBBADs-FTCD Recombinant Plasmid

PCR amplification was performed using plasmid pMD19-T-FTCD as a template. The amplification product was detected by 1% agarose gel electrophoresis, and a target fragment was recovered by cutting the gel; the target fragment and pBBADs vector were digested by double enzymes, BpiI and SpeI, followed by gel recovery and ligation at 16° C. overnight, and verification by sequencing.

8.3.3. Construction of Recombinant Lactobacillus by Electrotransformation

Electrocompetent Bifidobacterium longum was prepared by the method described by Reyes Escogidi, et al. Single colonies were randomly picked from the transformation plate and detected by colony PCR. Further sequencing was performed to verify that the sequence and the reading frame of the recombinant expression vector were correct.

8.3.4. Expression of the Target Protein in Bifidobacterium

Positive single colonies were picked and cultured in an MRS liquid medium for 24 h, diluted at 1:100 and then cultured to the logarithmic growth stage. The culture was induced to express proteins by adding L-arabinose to a final concentration of 0.2%, and cultured under an anaerobic condition at 37° C. for 5 to 6 h. The bacteria and the supernatant were collected and detected by SDS-PAGE. The results showed that FTCD was successfully expressed in Bifidobacterium.

8.4 Efficient Expression of the De-Epoxidase Gene of Epichloë in Saccharomyces cerevisiae

Unless specifically stated otherwise, the contents in this step is the same as in 8.1. The Saccharomyces cerevisiae expression vector pYES2-α was constructed by PCR amplification using the pPICZαA plasmid as a template to obtain the yeast signal peptide a factor, and cloning it into the Saccharomyces cerevisiae expression vector pYES2.

8.4.1. Construction of Recombinant Expression Vector pYES2-α-FTCD

The primers with EcoRI and XhoI restriction sites were designed according to the expression vector pYES2-α, and the primer sequences were as follows (underlined sequences indicate the restriction sites):

Forward primer:
5′-GCTGGAATTCATGGCCACCCCCACCTCCAC-3′
Reverse primer:
5′-CATGCTCGAGCTTCACCTCGGCATACTTGTC-3′

PCR amplification was performed using plasmid pMD19-T-FTCD as a template. The amplification product was detected by 1% agarose gel electrophoresis, and a target fragment was recovered by cutting the gel; the target fragment and pYES2-a vector were digested by double enzymes, EcoRI and XhoI, respectively, followed by gel recovery and ligation with T4 ligase; and the ligation product was mixed with DH5α. After mixing well, the mixture was placed in a water bath at 37° C. and left to stand for 30 to 60 min, and shaken at 37° C. and 220 r/min for 1 h. After shaking, the mixture of the recombinant plasmid and the competent cells were spread on an ampicillin-resistant medium and cultured overnight at 37° C.; single colonies were picked and identified by colony PCR and double digestion. Further sequencing was performed to verify that the sequence and the reading frame of the recombinant expression vector pYES2-α-FTCD were correct.

8.4.2. Construction of Recombinant Lactobacillus by Electrotransformation

Single colonies of Saccharomyces cerevisiae INVSc1 were picked and inoculated into 5 ml of YPD liquid medium for overnight culture at 30° C.; 1 ml of culture solution was taken and transferred to 40 ml of YPD medium and cultured to A600 of 0.8 to 1.0, and centrifuged at 3,500 rpm and 4° C. for 5 min, and the supernatant was discarded; the pellet was washed twice with sterile water pre-cooled with ice, washed once again with 1 M sorbitol pre-cooled with ice, and then suspended in 150 μL of 1 M sorbitol pre-cooled with ice. 1 μg of recombinant plasmid was added to 80 μL of pre-cooled competent cells, the mixture was placed in an ice bath for 5 min, and transferred to a 0.2 cm electrotransformation cup; the electroporation apparatus was provided with an electric field strength of 1.5 kV/cm, a capacitance of 25 μF, and a resistance of 100 to 200Ω; after electric shock, 1 ml of pre-cooled 1M sorbitol solution was quickly added to the mixture, mixed well and transferred to a 1.5 ml centrifuge tube, incubated at 30° C. for 1 h, and centrifuged at room temperature and 3,000 rpm for 5 min, and 800 μL of supernatant was discarded; the remaining culture was pipetted evenly, spread on the YPDS plate, and cultured at 30° C. for 2 to 4 days.

Single colonies were randomly picked from the transformation plate and detected by colony PCR. Further sequencing was performed to verify that the sequence and the reading frame of the recombinant expression vector pBBADs-FTCD were correct.

8.4.3. Expression of Polypeptides in Saccharomyces cerevisiae

The target protein was mainly secreted into the medium in a soluble state in Saccharomyces cerevisiae. The positive yeast cells comprising the recombinant plasmid were inoculated into 3 ml of YSD liquid medium, and cultured at 30° C. with shaking at 250 rpm for about 24 h until A600=2 to 6; 150 μL of the culture solution was taken and transferred into a fresh TPD medium and cultured at 30° C., 0.5 ml was taken every 24 h and centrifuged to collect the supernatant for SDS-PAGE detection. The results showed that FTCD can be secreted and expressed in Saccharomyces cerevisiae.

8.5 Efficient Expression of the De-Epoxidase Gene of Epichloë in Pichia pastoris and Verification

Unless specifically stated otherwise, the contents in this step is the same as in sections 5.1 to 5.3.

8.5.1. Construction of Pichia pastoris Expression Plasmid pPICZαA-FTCD

By designing the following primers, the deoxygenase gene derived from Epichloë was linked to EcoRI and XbaI restriction sites:

F:
5′-AGCTGAATTCATGGCCACCCCTACCTCCACCTC-3′
R:
5′-TTGTTCTAGATATTTAACTTCTGCATATTTATC-3′

The product was digested by double enzymes, EcoRI and XbaI, and meanwhile the expression vector pPICZαA was digested with these enzymes. The large fragment of the vector and the target gene fragment were recovered respectively, and the recovered fragments were ligated with T4 DNA ligase and transformed into Escherichia coli DH5α. After identification by colony PCR, the positive monoclonal bacterial solution was sequenced for verification.

8.5.2. Transformation of Pichia pastoris

The recombinant plasmids were first linearized with Sac I, and 1 ml of single-stranded DNA sample was boiled for 5 minutes and then rapidly cooled on ice. The samples were kept on ice. Competent yeast cells were centrifuged, and LiCl was removed with a pipette. 240 μl of 50% polyethylene glycol, 36 μl of 1 M LiCl, 25 μl of 2 mg/ml single-stranded DNAs, and plasmid DNAs (5 to 10 μg) in 50 μl of sterile water were sequentially added. Each tube was vortexed vigorously until the cell pellet was completely mixed (for about 1 minute). The test tubes were incubated at 30° C. for 30 minutes, and underwent a thermal shock in a water bath at 42° C. for 20 to 25 minutes. Cells were pelleted by centrifugation. The pellet was resuspended in 1 ml of YPD and incubated at 30° C. with oscillation. After 1 hour and 4 hours, 25 to 100 μl were inoculated on the YPD plates comprising an appropriate concentration of Zeocin™. The plates were incubated at 30° C. for 2 to 3 days.

10 single colonies were selected for enrichment culture, yeast chromosomal DNAs were extracted, and positive recombinant cells were detected by PCR. PCR identification was usually performed using pPICZαA universal primers. If the yeast expression vector pPICZαA was used as the template, a target fragment of about 588 bp can be amplified; and if pPICZαA-FTCD was used as the template, a target fragment with a target band size plus 588 bp can be amplified.

8.5.3. Enzyme Expression and Toxin Treatment

The screened positive yeast single colony (X33/pPICZαA-FTCD) and the negative yeast single colony (X33/pPICZαA) were respectively inoculated into 25 ml of BMGY medium, and cultured at 28° C. to 30° C. until OD600 was 2 to 6. The culture was centrifuged at room temperature, the supernatant was discarded, the cells were collected, the cells were resuspended in BMMY liquid medium to about OD600=1, transferred to a 500 ml Erlenmeyer flask, and cultured at 28° C. to 30° C., and methanol was added every 24 h to a final concentration of 0.5% to maintain induced expression. After 48 h of induction, the culture solution was aliquoted into 5 ml to 15 ml centrifuge tubes, and various trichothecenes were added to a final concentration of 25 μg/ml, the induction was continued for 48 h to 72 h, and the culture were collected for LC-HRMS analysis.

The results of LC-HRMS detection showed that transfer of the de-epoxidase gene into Pichia pastoris can achieve efficient catalysis of conversion of trichothecene mycotoxins (comprising DON, 3-DON, 15-ADON, FUS-X, NIV, T-2, HT-2, and DAS) to glutathione adducts. Transgenic yeast had improved ability of toxin tolerance, demonstrating that FTCD can take a trichothecene mycotoxin as a substrate and catalyze it into the corresponding GSH adduct, thereby playing a role in detoxification in vivo.

8.6 Analysis of the Content of Toxins in Feed Samples Treated with Multiple Strains.

8.6.1. Experimental Materials

30 feed samples were collected from Henan, Jiangsu and Anhui provinces, etc. DON, 3-ADON, 15-ADON, FUS-X, NIV, T-2 and HT-2 toxin standards, L-reduced glutathione (Sigma-Aldrich, USA), methanol (HPLC grade, CNW, Germany), acetonitrile (HPLC grade, CNW, Germany), and acetic acid (HPLC grade, Sigma-Aldrich, USA).

8.6.2. Experimental Methods

8.6.2.1 Analysis of Samples by LC-HRMS (/MS)

4 g of each of 30 feed samples was weighed, ground into powder, and dissolved in 1.3 ml of pre-cooled 75% methanol aqueous solution (comprising 0.1% formic acid). The mixture was vibrated for 10 s, sonicated for 30 min at room temperature, and the supernatant was taken and transferred to a new centrifuge tube. The supernatant was concentrated in vacuo to a dry powder. Before injection, the dry powder was resuspended with 100 μL of 20% acetonitrile solution, filtered through a 0.22 μm filter membrane, and transferred to an injection vial for LC-HRMS detection.

8.6.2.2 Treatment of Feed Samples with a Variety of Probiotics Comprising FTCD

According to the results of mass spectrometry, the one with the most serious contamination by a variety of trichothecene mycotoxins was selected as the sample to be treated. 30 g of the sample were weighed and ground into powder, 2 g of the sample was weighed and charged into a 15 ml centrifuge tube, 2 ml of PBS was added to prepare a powdery homogeneous solution, and then 5 ml of culture solution with OD600=0.8 of each of 5 kinds of transgenic probiotics comprising FTCD (in which Bifidobacterium was cultured under anaerobic conditions) and an appropriate amount of glutathione were added, and the treatment was performed in triplicate tubes for each kind of probiotic. A sample with an equal volume of PBS was used as a blank control. Treatment was performed at 25° C. for 2 h, and samples were taken at 0 h, 0.5 h, 1 h, and 2 h respectively for further LC-HRMS (/MS) analysis.

8.6.3. Experimental Results

The relative abundances of DON, 3-ADON, 15-ADON, NIV, T-2 and HT-2 toxins at 4 different time points of the feed treatment were quantified according to the PRM results of LC-HRMS. The results were shown in FIGS. 19A-19E, indicating that a variety of transgenic microorganisms comprising FTCD had a clearance effect on DON in highly processed products of maize.

In addition, the inventor had also verified that the protein as set forth in SEQ ID NO: 1 derived from Thinopyrum can be expressed in yeast cells to obtain an active protein.

IX. Detoxification Effect Test of Food and Beverage

1. Materials and Methods

Highly processed products of maize, i.e., spouting corn bran, spouting germ meal, and protein powder; Coca-Cola apple juice, and Huiyuan apple juice (an appropriate amount of a DON standard was added to adjust the content of DON in juice); and FTCD protein purified in vitro. Vomitoxin, and L-reduced glutathione (Sigma-Aldrich, USA).

2. Experimental Methods

2.1 Clearance of DON in Highly Processed Products of Maize by FTCD Protein Purified In Vitro

10 g of highly processed products of maize, i.e., spouting corn bran, spouting germ meal, and protein powder, were weighed respectively and ground into powder, 2 g of each sample was weighed and charged into a 15 ml centrifuge tube, 4 ml of PBS was added to prepare a powdery homogeneous solution, and then 100 μg of purified FTCD protein and an appropriate amount of glutathione were added, and the treatment was performed in triplicate tubes for each product. A sample with an equal volume of PBS was used as a blank control. Treatment was performed at 25° C. for 12 h, and samples were taken at 0 h, 1 h, 3 h, and 12 h respectively for further LC-HRMS (/MS) analysis.

2.2 Clearance of DON in Juice by FTCD Protein Purified In Vitro

1 ml of each of the two brands of juice was taken and charged into a 2 ml centrifuge tube, 25 μg of purified FTCD protein and an appropriate amount of glutathione were added, and the treatment was performed in triplicate tubes for each product. A sample with an equal volume of PBS was used as a blank control. Treatment was performed at 25° C. for 12 h, and samples were taken at 0 h, 1 h, 3 h, and 12 h respectively for further LC-HRMS (/MS) analysis.

3. LC-HRMS (/MS) Analysis

The in vitro reaction solutions of these products were centrifuged and filtered through 0.22 μm filter membranes, and transferred to injection vials for LC-HRMS detection.

3.1 Clearance Effect of FTCD Protein Purified In Vitro on DON in Highly Processed Products of Maize

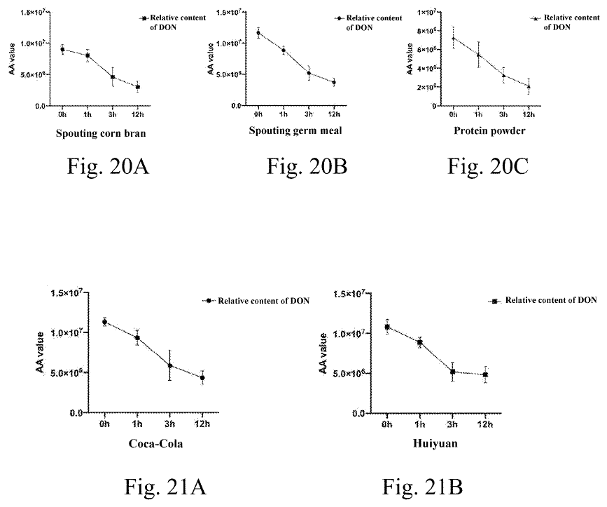

The relative abundances of DON toxins in highly processed products of maize before and after treatment were quantified according to the PRM results of LC-HRMS. The results were shown in FIGS. 20A-20C. The three kinds of highly processed products of maize, i.e., spouting corn bran, spouting germ meal, and protein powder, were treated with FTCD protein purified in vitro, and samples were taken at 0 h, 1 h, 3 h, and 12 h of treatment respectively for LC-HRMS analysis. It was found that the contents of DON in these three products gradually decreased over the treatment time, and the contents of DON in the products can be reduced by about 70% after treatment for 12 h.

3.1 Clearance of DON in Juice by FTCD Protein Purified In Vitro

The relative abundances of DON toxins in two brands of apple juice samples before and after treatment were quantified according to the PRM results of LC-HRMS. The results were shown in FIGS. 21A and 21B. The Coca-Cola apple juice and Huiyuan apple juice (an appropriate amount of a DON standard was added to adjust the content of DON in juice) were treated with FTCD protein purified in vitro, and samples were taken at 0 h, 1 h, 3 h, and 12 h of treatment respectively for LC-HRMS analysis. It was found that the contents of DON in these three products gradually decreased over the treatment time, and the contents of DON in the products can be reduced by about 50% after treatment for 12 h.

The highly processed products of maize, i.e., spouting corn bran, spouting germ meal, and protein powder, as well as Coca-Cola apple juice and Huiyuan apple juice (an appropriate amount of a DON standard was added to adjust the content of DON in juice) were treated with FTCD protein purified in vitro, and the results were analyzed by LC-HRMS detection, indicating that the protein had good detoxification capability of vomitoxin in a variety of products, which further proved its important practical application value.

Although the invention has been described with reference to the exemplary embodiments, it should be understood that the invention is not limited to the disclosed exemplary embodiments. Without departing from the scope or spirit of the invention, various adjustments or changes can be made to the exemplary embodiments of the present specification. The scope of the claims should be based on the broadest interpretation to cover all modifications and equivalent structures and functions.

1-39. (canceled)

40. An isolated polypeptide having epoxy group-removing catalytic activity, wherein the polypeptide is capable of catalyzing a reaction between an epoxy group of a trichothecene mycotoxin and glutathione in a PBS buffer at a temperature of 15° C. to 35° C. to produce a glutathionylated derivative.

41. An isolated polypeptide having epoxy group-removing catalytic activity, comprising an amino acid sequence selected from the group consisting of the following (1) to (5):
(1) an amino acid sequences as set forth in any of SEQ ID Nos: 1-35;
(2) an amino acid sequence which has 85% or more sequence identity with the amino acid sequence of (1) and is derived from the same genus;
(3) an amino acid sequence which has one or more amino acid mutations and has 85% or more sequence identity as compared with the amino acid sequence of (1) or (2), and still maintains the original epoxy group-removing catalytic activity;
(4) a partial consecutive sequence, preferably an N-terminal partial consecutive sequence, derived from the amino acid sequence of any of (1) to (3), and still having the original polypeptide activity; and
(5) a chimeric sequence in which an additional amino acid sequence is linked to the N-terminal and/or C-terminal of the amino acid sequence of any of (1) to (4).

42. The polypeptide according to claim 40, wherein the polypeptide is derived from Thinopyrum or Epichloë.

43. The polypeptide according to claim 41, wherein the polypeptide has conserved sites of amino acid A at position 98 and amino acid A at position 99, when the amino acid sequence as set forth in SEQ ID NO: 1 is used as a positional reference.

44. An isolated active polypeptide having an amino acid sequence of:
V1-GDX1X2DIAAX3LQRT-V2-ADYARFNX1NVDX4AFX5AHV
X1X6MX6HGLPLDPAX7X4DVX8KAEFVR-V3,
wherein:
X1 represents G or S; X2 represents F or L; X3 represents Y or H; X4 represents A or V; X5 represents T or Q or N; X6 represents L or V; X7 represents T or S; and X8 represents T or I;
V1 is absent or represents a first variable region, the amino acid sequence of the first variable region corresponds to a sequence of a plurality of consecutive amino acids before the amino acid at position 92 in SEQ ID NO: 1, and the sequence identity of the first variable region with the sequence of the plurality of consecutive amino acids is 85% or more;
V2 represents a linker arm or represents a second variable region, the amino acid sequence of the second variable region corresponds to a sequence of a plurality of consecutive amino acids between the amino acids at positions 105 to 143 in SEQ ID NO: 1, and the sequence identity of the second variable region with the sequence of the plurality of consecutive amino acids is 85% or more; and
V3 is absent or represents a third variable region, the amino acid sequence of the third variable region corresponds to a sequence of a plurality of consecutive amino acids after the amino acid at position 144 in SEQ ID NO: 1, and the sequence identity of the third variable region with the sequence of the plurality of consecutive amino acids is 85% or more.

45. An isolated nucleic acid molecule encoding the polypeptide according to claim 40.

46. An isolated nucleic acid molecule having a base sequence selected from the group consisting of the following (a) to (e):
(a) a sequence as set forth in any of SEQ ID Nos: 36-70;
(b) a sequence modified for the host codon bias based on the base sequence of (a);
(c) a conserved region sequence of the sequences as set forth in (a);
(d) a sequence which has 85% or more sequence identity with any of (a) to (c), is derived from the same genus, and encodes a polypeptide having epoxy group-removing catalytic activity; and
(e) a sequence complementary to at least a portion of any of the sequences of (a) to (d).

47. A nucleic acid construct, comprising the nucleic acid according to claim 45 and optionally a regulatory element.

48. A pharmaceutical composition for detoxification, comprising a polypeptide having epoxy group-removing activity or a nucleic acid encoding therefor and optionally a pharmaceutically acceptable carrier, wherein the active polypeptide is capable of catalyzing a reaction between a trichothecene mycotoxin and glutathione to produce a glutathionylated derivative, thereby removing epoxy groups that cause toxin toxicity.

49. The pharmaceutical composition for detoxification according to claim 48, wherein the active polypeptide is the polypeptide, and the nucleic acid is the nucleic acid molecule.

50. The pharmaceutical composition for detoxification according to claim 48, further comprising glutathione.

51. A food and beverage or feed composition, comprising de-epoxidase which is capable of catalyzing a reaction between a trichothecene mycotoxin and glutathione in a PBS buffer at a temperature of 15° C. to 35° C. to produce a glutathionylated derivative.

52. The food and beverage or feed composition according to claim 51, wherein the enzyme is the polypeptide.

53. The food and beverage or feed composition according to claim 51, wherein the food and beverage composition further comprises glutathione.

54. The food and beverage or feed composition according to claim 51, wherein the food and beverage or feed composition comprises at least one grain flour selected from the group consisting of wheat flour, barley flour, rye flour, oat flour, corn flour, millet flour, rice flour, sorghum flour, soybean flour, potato flour, sweet potato flour, and peanut flour; or comprises a processed food product such as soybean hull, soybean milk, germ meal, germ, vegetable oil, starch, glucose, protein powder, alcohol and fermented product.

55. The food and beverage or feed composition according to claim 51, comprising at least one fruit juice or beverage ingredient selected from the group consisting of milk, dairy products, apples, citruses and grapes.

56. An engineered microorganism, comprising a nucleic acid derived from Thinopyrum and/or Epichloë that is introduced exogenously or by gene recombination, the nucleic acid being the nucleic acid according to claim 45.

57. The engineered microorganism according to claim 56, comprising the polypeptide.

58. A method for catalyzing a reaction of removing an epoxy group of a trichothecene, wherein the method comprises contacting the polypeptide according to claim 40 with a trichothecene and glutathione, thereby producing a glutathionylated derivative; preferably, the trichothecene comprises deoxynivalenol, 15-acetyl-deoxynivalenol, 3-acetyl-deoxynivalenol, nivalenol, fusarenon-X, diacetoxyscirpenol, T-2 toxin and HT-2 toxin.

59. A method for preventing cell poisoning or relieving cytotoxicity, wherein the method comprises contacting a cell to be treated with a polypeptide having epoxy group-removing activity or a nucleic acid encoding therefor, or a cell producing the active polypeptide, and optionally glutathione.

60. A method for reducing or decreasing a toxin in a composition, wherein the method comprises contacting a food and beverage or feed raw material comprising a toxin with de-epoxidase or a cell producing the enzyme under conditions suitable for the reaction, wherein the toxin is a trichothecene.

61. A glutathionylated derivative, having a structure shown in the following general formula (I): 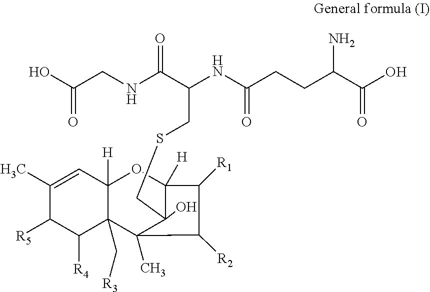
wherein each of R₁, R₂ and R₃ independently represents a hydrogen atom, a hydroxyl group or an ester group represented by —OCO—R′, wherein R′ is a linear or branched C₁-C₈ alkyl group, R₄ represents a hydrogen atom or a hydroxyl group, and R₅ represents a hydrogen atom, ═O, a hydroxyl group or an ester group represented by —OCO—R″, wherein R″ is a linear or branched C₁-C₁₀ alkyl group.

62. A method for plant breeding, disease control comprising:
introducing the nucleic acid according to claim 45 into a host;
allowing the nucleic acid to be expressed, thereby obtaining a polypeptide having epoxy group-removing activity;
degrading toxins by using the polypeptide to generate glutathionylated derivative.
